(19) United States
(12) Patent Application Publication
(10) Pub. No.: US 2002/0091090 A1
(43) Pub. Date: Jul. 11, 2002

(54) Somatostatin antagonists and agonists

(76) Inventors: Bridget M. Cole, Stonington, CT (US); Anthony P. Ricketts, Stonington, CT (US); Bruce A. Hay, East Lyme, CT (US)

Correspondence Address: Paul H. Ginsburg, Pfizer Inc, 20th Floor, 235 East 42nd Street, New York, NY, 10017-5755, US

(21) Appl. No.: 09/952,300

(22) Filed: Sep. 14, 2001

Related U.S. Application Data
(60) Provisional application No. 60/258,799, filed on Dec. 28, 2000.

(51) Int. Cl. A61K 38/05; C07D 413/02; C 07D 4 3/14; C 07D 4 3/02

(52) U.S. Cl. 514/19; 514/254.09; 514/323; 514/419; 544/373; 540/547; 546/199; 546/201; 548/455

(57) ABSTRACT

Compounds according to the formula A-B-Z-W, wherein

A is selected from (C₆-C₁₀)aryl-, or (C₁-C₉)heteroaryl-, which groups may be optionally substituted;

B is selected from

(a) O, NH, NR¹⁰, —(CH₂)ₖ—O—, —(CH₂)ₖ—N—, and —(CH₂)ₖ—NR¹⁰—, where R¹⁰is (C₁-C₆)alkyl and where k is 1 to 6 in each case, or

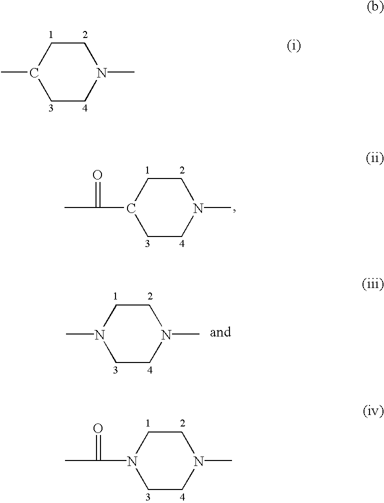

where said group (i) through (iv) is optionally substituted by 1 to 4, preferably 1 to 2, groups selected from (C₁-C₆)alkyl, halo, and (C₁-C₆)alkyl optionally substituted by 1 to 3 halo atoms wherein one of carbon atoms C₁, C₂, C₃ and C₄ of said piperidine or piperazine group is optionally replaced by a carbonyl group;

Z and W are as herein described; and pharmaceutically acceptable salts, solvates or hydrates thereof; pharmaceutical compositions thereof; and methods useful to facilitate secretion of growth hormone(GH) in mammels.

### FIELD OF THE INVENTION

The present invention provides pharmaceutically active compounds that facilitate secretion of growth hormone (GH) by the anterior pituitary. Growth hormone (also known as somatotropin) acts indirectly to promote skeletal growth in children by stimulating the production of insulin like growth factor-1 from the liver. Growth hormone also stimulates the differentiation of fat cells and chondrocytes (cells that secrete collagen and proteoglycans to form cartilage). In adults, growth hormone is involved in the proper maintenance of connective and muscle tissues.

Growth hormone deficiency may be congenital or acquired. Deficiency in children causes slow skeletal growth that, if not corrected, results in permanent short stature. In older adults, deficiency of growth hormone results in frailty. Additional adult symptoms of GH deficiency may include wrinkled skin and hypoglycemia.

For veterinary application, upregulation of growth hormone is useful to treat frailty in older animals, particularly companion animals. With respect to livestock, upregulation of growth hormone increases growth and performance, even in healthy animals with normal GH levels. Improvements in feed efficiency, milk yield, leanness, meat quality and fertility are of note.

Although direct administration of growth hormone may be effective in certain therapeutic applications, it is difficult in practice. Among other issues, since the half-life of growth hormone in the body is very short, direct administration leads to artificially increased levels in the concentration of circulating GH, which then rapidly drop off. Sustained release, such as by a mechanical pump, has not been optimally set to practice.

The concentration of growth hormone circulating in the body depends on the balance of numerous biochemical pathways, including opposing processes. Compared to the direct administration approach, shifting the balance of these pathways indirectly provides a safer, more reproducible method to affect GH secretion on a sustained basis. Under this approach, since the overall regulatory framework remains intact, secretion rates and circulatory concentrations for GH follow a relatively normal pattern, and adverse fluctuations in both secretion rate and circulating GH concentration are avoided. The present invention provides for therapeutic compounds, and their use, to indirectly elevate growth hormone secretion from the pituitary.

### Reported Developments

Growth hormone is released from the anterior pituitary in response to stimulation by growth hormone releasing peptide (GHRP), and growth hormone releasing hormone (GHRH), of hypothalmic origin. However, release of growth hormone via these or other mechanisms is inhibited by somatostatin, and thus the process is closely regulated.

Somatostatin (SRIF) is a cyclic peptide hormone of 14 amino acids (there is also a 28 amino acid form) having numerous endocrine functions which, like many hormones, is cleaved from a larger precursor protein. Somatostatin inhibits the pituitary secretion of growth hormone, the pancreatic secretion of glucagon and insulin, and the secretion of gastrin from the gut. Somatostatin also acts as a neurotransmitter/neuromodulator (see S. J. Hocart et al., J. Med. Chem., 41, pp. 1146-1154, 1998 for general discussion).

The biological effects of somatostatin are apparently all inhibitory in nature, and are elicited upon binding to the surface of a target cell. The receptor is an integral membrane protein (which spans the cell membrane), and is G-protein-coupled. G-protein coupled receptors represent a major class of cell surface receptors. It is believed that upon binding of somatostatin to the receptor, the receptor undergoes a conformational change facilitating its interaction with a G-protein at the cytoplasmic face of the receptor. This facilitates binding or release of GTP/GDP at the G protein, and leads to further activation and signalling events inside the cell. In particular, somatostatin binding at its own G-protein-coupled receptor is negatively coupled to adenylyl cyclase activity, which is necessary for the production of cylic AMP. Thus, these further signalling events directly oppose mechanisms (for example, as mediated by calcium ions or cyclic AMP) whereby GHRP and GHRH would otherwise trigger extracellular secretion of growth hormone from cytoplasmic storage granules. For a general review thereof, see The Encyclopedia of Molecular Biology, J. Kendrew, ed., Blackwell Science, Ltd. 1994, at page 387.

The effects of somatostatin on target cells are mediated by at least 5 classes of receptors (sst1-sst5). Although the receptors may have similar affinity for somatostatin, they are differentially expressed in different tissues, and so positioned, interact, directly or indirectly, with different intracellular signalling components. This tissue specificity of receptor expression accounts in large measure for the different effects of somatostatin in different target cell types. Somatostatin receptors are found, for example, in tissues of the anterior pituitary, other brain tissues, the pancreas, the lung, on lymphocytes, and on mucosa cells of the intestinal tract.

The sst2 type receptor is known to mediate inhibition of growth hormone secretion in the anterior pituitary. This receptor is also reported in 2 forms, proteins sst2A and sst2B, which result from differential splicing of the sst2 gene transcript (M. Vanetti, et al., FEBS Letters, 311, pp.290-294, 1992). The sst2 receptor is also known to mediate inhibition of gastrin and histamine secretion. Additionally, the sst2 receptor is known to mediate inhibition of glucagon release from pancreatic alpha cells.

Although numerous somatostatin agonists have been described (see for example, WO 98/44922, WO 98/45285, and WO 98/44921), the development of useful sst2-linked somatostatin antagonists has lagged behind. Recent reports of such compounds include W. R. Baumbach et al., Molecular Pharmacology, 54, pp. 864-873, 1998, and S. J. Hocart et al., J. Med. Chem., 41, pp. 1146-1154, 1998. However, such compounds are short peptides, a class of molecules not often suited for successful use as pharmaceuticals because of their typically short half life in the body. Additional relevant disclosures include WO99/64401 and WO99/64420.

It would be advantageous to provide antagonists of somatostatin activity, effective at the sst2 type receptor, having superior properties as pharmaceuticals, including bioavailability, stability, and the like. The present invention provides a series of antagonist compounds that specifically interfere with the binding of somatostatin to the sst subtype 2 receptors of cells in the mammalian anterior pituitary, and which have additional valuable properties.

### SUMMARY OF THE INVENTION

According to formula (I),

A-B-Z-W

wherein

A is selected from

(a) (C₆-C₁₀)aryl-, selected from phenyl or naphthyl; or

(b) (C₁-C₉)heteroaryl-, selected from the group consisting of furyl-, thienyl-thiazolyl-, pyrazolyl-, isothiazolyl-, oxazolyl-, isoxazolyl-, pyrrolyl-, triazolyl-, tetrazolyl-, imidazolyl-, 1,3,5-oxadiazolyl-, 1,2,4-oxadiazolyl-, 1,2,3-oxadiazolyl-, 1,3,5-thiadiazolyl-, 1,2,3-thiadiazolyl-, 1,2,4-thiadiazolyl-, pyridyl-, pyrimidyl-, pyrazinyl-, pyridazinyl-, 1,2,4-triazinyl-, 1,2,3-triazinyl-, 1,3,5-triazinyl-, pyrazolo[3,4-b]pyridinyl-, cinnolinyl-, pteridinyl-, purinyl-, 6,7-dihydro-5H-[1]pyrindinyl-, benzo[b]thiophenyl-, 5,6,7,8-tetrahydro-quinolin-3-yl, benzoxazolyl-, benzothiazolyl-, benzisothiazolyl-, benzisoxazolyl-, benzimidazolyl-, thianaphthenyl-, isothianaphthenyl-, benzofuranyl-, isobenzofuranyl-, isoindolyl-, indolyl-, indolizinyl-, indazolyl-, isoquinolyl- quinolyl-, phthalazinyl-, quinoxalinyl-, quinazolinyl-, and benzoxazinyl-;

wherein said A group (a) or (b) is optionally substituted by zero to seven, preferably zero to five groups, each independently selected from:

hydroxy, halo, amino, trifluoromethyl-, carboxy, (C₁-C₆)alkoxy-, (C₁-C₆)acyloxy-, (C₁-C₆)alkylamino-, ((C₁-C₆)alkyl)₂amino-, (C₁-C₆)acylamino-, cyano, nitro, (C₁-C₆)alkyl-, (C₂-C₆)alkenyl-, (C₂-C₆)alkynyl-, (C₁-C₆)acylamino-, cyano(C₁-C₆)alkyl-, trifluoromethyl(C₁-C₆)alkyl-, nitro(C₁-C₆)alkyl-, (C₁-C₃)alky](difluoromethylene)(C₁-C₃)alkyl-, (C₁-C₆)acylamino(C₁ -C₆)alkyl-, (C₁-C₆)alkoxy(C₁-C₆)acylamino-, amino(C₁-C₆)acyl-, amino(C₁-C₆)acyl(C₁-C₆)alkyl-, (C₁-C₆)alkylamino(C₁-C₆)acyl-, ((C₁-C₆)alkyl)₂amino(C₁-C₆)acyl-, (C₃-C₁₀)cycloalkyl(C₁-C₆)alkyl-, (C₁-C₆)acyloxy(C₁-C₆)alkyl-, (C₂-C₆)alkoxy(C₁-C₆)alkyl-, piperazinyl(C₁-C₆)alkyl-, (C₁-C₆)acylamino(C₁-C₆)alkyl-, (C₆-C₁₀)ary](C₁-C₆)alkoxy(C₁-C₆)alkyl-, (C₁-C₉)heteroaryl(C₁-C₆)alkoxy(C₁-C₆)alkyl-, (C₁-C₆)alkylthio(C₁-C₆)alkyl-, (C₆-C₁₀)arylthio(C₁-C₆)alkyl-, (C₁-C₆)alkylsulfinyl(C₁-C₆)alkyl-, (C₆-C₁₀)arylsulfinyl(C₁-C₆)alkyl-, (C₁-C₆)alkylsulfonyl(C₁-C₆)alkyl-, (C₆-C₁₀)arylsulfonyl(C₁-C₆)alkyl-, amino(C₁-C₆)alkyl-, (C₁-C₆)alkylamino(C₁-C₆)alkyl-, (C₁-C₆)alkyl(difluoromethylene)-, (C₁-C₃)alkyl(difluoromethylene)(C₁-C₃)alkyl-, (C₁-C₆)alkoxy(C₁-C₆)acyl-, (C₁-C₆)alkylamino(C₁-C₆)acyl-, ((C₁-C₆)alkyl)₂amino(C₁-C₆)acyl-, (C₆-C₁₀)aryl-, (C₁-C₉(C₆-C₁₀)aryl-,(C₆-C₁₀)aryl(C₆-C₁₀)aryl(C₁-C₆)alkyl- (C₃-C₁₀)cycloalkyl-, (C₃-C₆)cycloalkyl(C₁-C₆)alkyl-, (C₃-C₁₀)heterocycloalkyl-, (C3-C₁₀)heterocycloalkyl(C₁-C₆)alkyl-, hydroxy(C₂-C₆)alkyl-, (C₁-C₆)acyloxy(C₂-C₆)alkyl-, (C₁-C₆)alkoxy(C₂-C₆)alkyl-, piperazinyl(C₁-C₆)alkyl-, (C₁-C₆)acylamino(C₁-C₆)alkyl-, (C₆-C₁₀)aryl(C₁-C₆)alkoxy(C₁-C₆)alkyl-, (C₁-C₉)heteroaryl(C₁-C₆)alkoxy(C₁-C₆)alkyl-, (C₁-C₆)alkylthio(C₁-C₆)alkyl-, (C₆-C₁₀)arylthio(C₁-C₆)alkyl-, (C₁-C₆)alkylsulfinyl(C₁-C₆)alkyl-, (C₆-C₁₀)arylsulfinyl(C₁-C₆)alkyl-, (C₁-C₆)alkylsulfonyl(C₁-C₆)alkyl-, (C₆-C₁₀)arylsulfonyl(C₁-C₆)alkyl-, amino(C₁-C₆)alkyl-, (C₁-C₆)alkylamino(C₁-C₆)alkyl-, and ((C₁-C₆)alkyl)₂ amino(C₁-C₆)alkyl-;

B is selected from

(a) O, NH, NR¹⁰, —(CH₂)ₖ—O—, —(CH₂)ₖN—, and —(CH₂)ₖ—NR¹⁰—, where R¹⁰ is (C₁-C₆)alkyl, and where k is 1 to 6 in each case, or

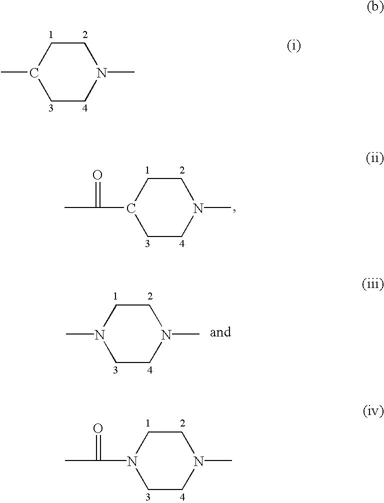

where said group (i) through (iv) is optionally substituted by 1 to 4, preferably 1 to 2, groups selected from (C₁-C₆)alkyl, halo, and (C₁-C₆)alkyl optionally substituted by 1 to 3 halo atoms, wherein one of carbon atoms C₁, C₂, C₃ and C₄ of said piperidine or piperazine group is optionally replaced by a carbonyl group;

Z is selected from groups (i) to (xiv):

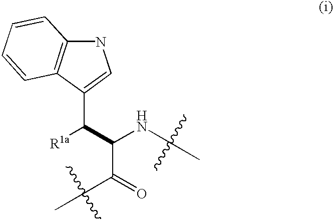

where R^{1a} is H, or (C₁-C₆)alkyl optionally substituted by 1 to 3 halo atoms;

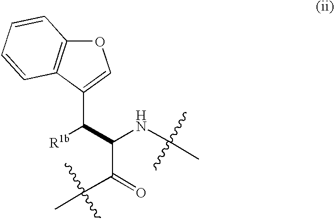

where R^{1b} is H, or (C₁-C₆)alkyl optionally substituted by 1 to 3 halo atoms;

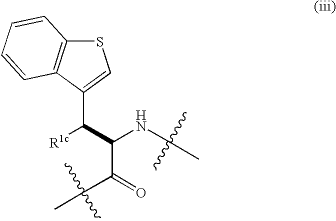

where R^{1c} is H, or (C₁-C₆)alkyl optionally substituted by 1 to 3 halo atoms;

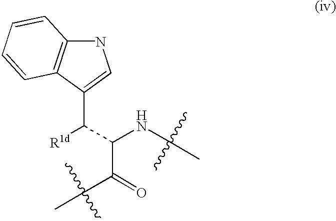

where R^{1d} is H, or (C₁-C₆)alkyl optionally substituted by 1 to 3 halo atoms;

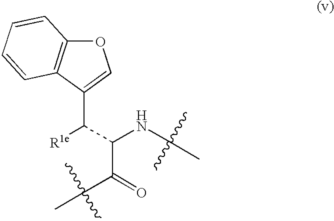

where R^{1e} is H, or (C₁-C₆)alkyl optionally substituted by 1 to 3 halo atoms;

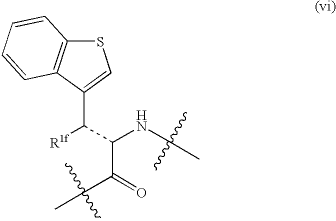

where R^{1f} is H, or (C₁-C₆)alkyl optionally substituted by 1 to 3 halo atoms;

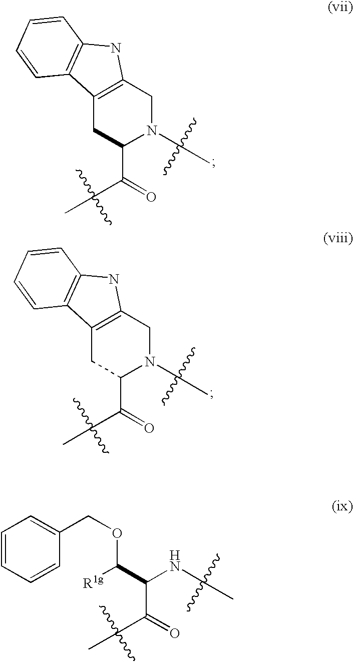

where R^{1g} is H, or (C_{1-C}₆)alkyl optionally substituted by 1 to 3 halo atoms;

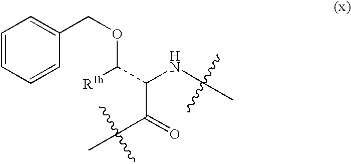

where R^{1h} is H, or (C₁-C₆)alkyl optionally substituted by 1 to 3 halo atoms;

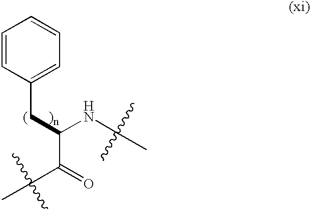

where n is 0 to 6, preferably 1 to 3;

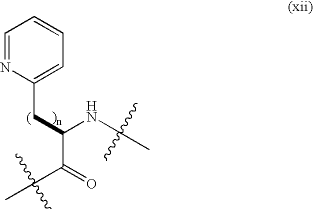

where n is 0 to 6, preferably 1 to 3;

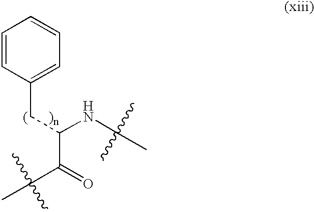

where n is 0 to 6, preferably 1 to 3; and

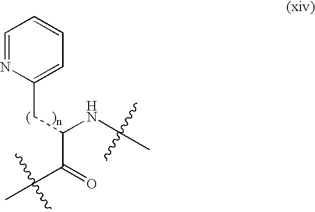

where n is 0 to 6, preferably 1 to 3; and

W is selected from:

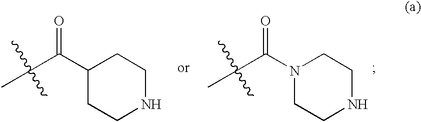

wherein one or more ring carbons of said piperidine or piperazine ring is optionally substituted by (C₁-C₆)alkyl or halo;

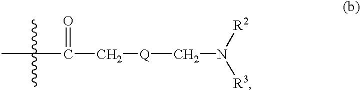

wherein Q is selected from the group consisting of:

(i) (C₆-C₁₀)aryl-, selected from phenyl or naphthyl;

(ii) (C₁-C₉)heteroaryl-, selected from the group consisting of furyl-, thienyl-thiazolyl-, pyrazolyl-, isothiazolyl-, oxazolyl-, isoxazolyl-, pyrrolyl-, triazolyl-, tetrazolyl-, imidazolyl-, 1,3,5-oxadiazolyl-, 1,2,4-oxadiazolyl-, 1,2,3-oxadiazolyl-, 1,3,5-thiadiazolyl-, 1,2,3-thiadiazolyl-, 1,2,4-thiadiazolyl-, pyridyl-, pyrimidyl-, pyrazinyl-, pyridazinyl-, 1,2,4-triazinyl-, 1,2,3-triazinyl-, 1,3,5-triazinyl-, pyrazolo[3,4-b]pyridinyl-, cinnolinyl-, pteridinyl-, purinyl-, 6,7-dihydro-5H-[1]pyrindinyl-, benzo[b]thiophenyl-, 5,6,7,8-tetrahydro-quinolin-3-yl, benzoxazolyl-, benzothiazolyl-, benzisothiazolyl-, benzisoxazolyl-, benzimidazolyl-, thianaphthenyl-, isothianaphthenyl-, benzofuranyl-, isobenzofuranyl-, isoindolyl-, indolyl-, indolizinyl-, indazolyl-, isoquinolyl- quinolyl-, phthalazinyl-, quinoxalinyl-, quinazolinyl-, and benzoxazinyl-;

(iii) (C₃-C₁₀)cycloalkyl that is selected from the group consisting of cyclopropyl-, cyclobutyl-, cyclopentyl-; cyclohexyl-, cycloheptyl-, cyclopropenyl-, cyclobutenyl-cyclopentenyl-, cyclohexenyl-, cycloheptenyl-, 1,3-cyclobutadienyl-, 1,3-cyclopentadienyl-, 1,3-cyclohexadienyl-, 1,4-cyclohexadienyl- 1,3-cycloheptadienyl-, 1,4-cycloheptad ienyl-, 1,3,5-cycloheptatrienyl- bicyclo[3.2.1]octane, bicyclo [2.2.1] heptane and the norborn-2-ene unsaturated form thereof; and

(iv) (C₃-C₁₀)heterocycloalkyl that is selected from the group consisting of pyrrolidinyl-, tetrahydrofuranyl- dihydrofuranyl-, tetrahydropyranyl-, pyranyl-, thiopyranyl-, aziridinyl-, oxiranyl-, methylenedioxyl-, chromenyl-, isoxazolidinyl-, 1,3-oxazolidin-3-yl-isothiazolidinyl-, 1,3-thiazolidin-3-yl-, 1,2-pyrazolidin-2-yl-, 1,3-pyrazolidin-1-yl-, piperidinyl-, thiomorpholinyl-, 1,2-tetrahydrothiazin-2-yl-, 1,3-tetrahydrothiazin-3-yl-, tetrahydrothiadiazinyl-, morpholinyl-, 1,2-tetrahydrodiazin-2-yl-, 1,3-tetrahydrodiazin-1-yl-, tetrahydroazepinyl-, piperazinyl-, and chromanyl;

wherein R² and R³ thereof are each independently selected from H, (C₁-C₈)alkyl, and phenyl(CH₂)- , wherein said alkyl and phenyl groups are optionally substituted by one or more halo atoms; and

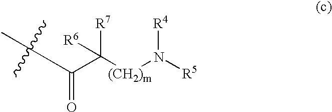

wherein m is 1 to 7, preferably 4 to 5;

R⁴ and R⁵ are each independently selected from the group consisting of

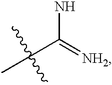

H, (C₁-C₆)alkyl, and phenyl(CH₂)—, wherein said alkyl and phenyl groups are optionally substituted by one or more halo atoms, and

R⁶ and R⁷ are each independently selected from

(i) H, CH₃—, NH₂—, and CH₃C(O)—NH—, or from

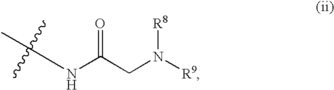

wherein R⁸ and R⁹ are each independently selected from H, (C₁-C₈)alkyl, and phenyl(CH₂)- , and said alkyl and phenyl groups are optionally substituted by one or more halo atoms.

With respect to the selection of groups R⁴, R⁵, R⁶, R⁷, R⁸ and R⁹ according to the above formulas, the practitioner of the art will immediately recognize that some combinations of said R groups would not be preferred, as they are unstable or difficult to prepare. For example, the simultaneous use of two amidine groups as R⁴ and R⁵ is not preferred. Similar non-preferred structures include use of two amino groups, or use of two acetamide groups, at R⁶ and R⁷, or the use of two structures of option (ii) in this regard.

With respect to group Z, it is preferred that any of R^{1(a-h)} be selected from H, methyl and trifluoromethyl.

In a preferred embodiment of the invention, W is option (c) thereof, wherein R⁶ is, for example, hydrogen, and R⁷ is amino. In this case, preferably W group defines an L-lysine group In further preferred examples, the W group is L-diaminopimelic acid, L-canavanine, L-ornithine, L-2,4-diaminobutyric acid, L-5-hydroxylysine, or L-epsilon-N-methyllysine. In further preferred examples, the W group is an L-arginine group.

In an additional embodiment of the invention, group W according to formula (c) is replaced by L-histidine or a derivative thereof such as L-3-methylhistidine, even though such structures are not derivable from the depicted formula.

In a further preferred embodiment of the invention, W is option (c) thereof, wherein RB is, for example, hydrogen, and R⁷ is provided by

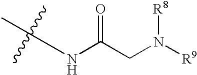

wherein each of R8 and R⁹ is independently selected from hydrogen and methyl. According to this embodiment of the invention, group W (c) becomes an amino acid such as lysine or arginine, in amide linkage to glycine, or a derivative thereof.

Additionally, where substitution by one or more halo atoms is permitted at any place aforementioned in the compounds of the invention, a preferred example is by one or two halo atoms. Preferably the halo atom(s) is selected from chlorine and fluorine. Generally speaking, trifluoromethyl is the preferred species of trifluoro(C₁-C₆)alkyl group. Where substitution by one or more trifluoromethyl groups is permitted, it is preferred that only a single trifluoromethyl group be incorporated.

Preferred compounds of the invention include:

7-Amino-heptanoic acid {2-(1 H-indol-3-yl)-1 -[2-(1H-indol-3-yl)-ethylcarbamoyl]-ethyl}-amide;

7-Amino-heptanoic acid [1 -[2-(5-fluoro-1H-indol-3-yl)-1-methyl-ethylcarbamoyl]-2-(1H-indol-3-yl)-ethyl]-amide;

7-Amino-heptanoic acid [1 -[2-(5-fluoro-1H-indol-3-yl)-ethylcarbamoyl]-2-(1H-indol-3-yl)-ethyl]-amide;

7-Amino-heptanoic acid (2-(1H-indol-3-yl)-1-{2-[2-(4-methoxy-phenyl)-1H-indol-3-yl]-ethylcarbamoyl}-ethyl)-amide;

7-Amino-heptanoic acid [1-(indan-2-ylcarbamoyl)-2-(1H-indol-3-yl)-ethyl]-amide;

7-Amino-heptanoic acid {1-(1H-indol-3-ylmethyl)-2-oxo-2-[4-(2-oxo-2,3-dihydro-benzoimidazol-1-yl)-piperidin-1-yl]-ethyl}-amide;

7-Amino-heptanoic acid [1-[2-(6-fluoro-1H-indol-3-yl)-ethylcarbamoyl]-2-( 1H-indol-3-yl)-ethyl]-amide;

7-Amino-heptanoic acid [2-[4-(2-chloro-dibenzo[b,f][1,4]oxazepin-11-yl)-piperazin-1-yl]-1-(1H-indol-3-ylmethyl)-2-oxo-ethyl]-amide;

6-Amino-hexanoic acid {1-(1H-indol-3-ylmethyl)-2-oxo-2-[4-(2-oxo-2,3-dihydro-benzoimidazol-1-yl)-piperidin-1-yl]-ethyl}-amide;

7-Amino-heptanoic acid (2-(1H-indol-3-yl)-1-{[2-(1H-indol-3-yl)-ethyl]-methyl-carbamoyl}-ethyl)-amide;

7-Amino-heptanoic acid {2-(1H-indol-3-yl)-1-[2-(1H-indol-3-yl)-ethylcarbamoyl]-ethyl}-amide;

7-Amino-heptanoic acid [1-[2-(6-benzyloxy-1H-indol-2-yl)-ethylcarbamoyl]-2-(1H-indol-3-yl)-ethyl]-amide;

6-Amino-hexanoic acid [1-(4-ethyl-benzylcarbamoyl)-2-(1H-indol-3-yl)-ethyl]-amide;

1-{1-[2-(7-Amino-heptanoyl)-2,3,4,9-tetrahydro-1H-carboline-3-carbonyl]-piperidin-4-yl}-1,3-dihydro-benzoimidazol-2-one;

7-Amino-heptanoic acid (2-(1H-indol-3-yl)-1-{[2-(1H-indol-3-yl)-ethyl]-methyl-carbamoyl}-ethyl)-amide;

7-Amino-heptanoic acid {2-(1H-indol-3-yl)-1-[2-(5-methoxy-1H-indol-3-yl)-ethylcarbamoyl]-ethyl}-amide;

7-Amino-heptanoic acid [1-[4-(biphenyl-4-carbonyl)-piperazine-1-carbonyl]-2-(1H-indol-3-yl)-ethyl]-amide;

7-Amino-heptanoic acid [2-(1H-indol-3-yl)-1-(4-phenyl-butylcarbamoyl)-ethyl]-amide;

7-Amino-heptanoic acid [1-[2-(2-fluoro-phenyl)-ethylcarbamoyl]-2-(1H-indol-3-yl)-ethyl]-amide;

7-Amino-heptanoic acid {1-(1H-indol-3-ylmethyl)-2-oxo-2-[4-(2-oxo-2,3-dihydro-benzoimidazol-1-yl)-piperidin-1-yl]-ethyl}-amide;

7-Amino-heptanoic acid [2-(1H-indol-3-yl)-1-(1H-indol-5-ylcarbamoyl)-ethyl]-amide;

7-Amino-heptanoic acid [2-(1H-indol-3-yl)-1-(3-phenyl-propylcarbamoyl)-ethyl]-amide;

2-(7-Amino-heptanoylamino)-3-(1H-indol-3-yl)-propionic acid biphenyl-4-ylmethyl ester;

7-Amino-heptanoic acid [2-(1H-indol-3-yl)-1-(2-phenyl-cyclopropylcarbamoyl)-ethyl]-amide;

6-Amino-hexanoic acid [1-(3-ethyl-benzylcarbamoyl)-2-(1H-indol-3-yl)-ethyl]-amide;

7-Amino-heptanoic acid {2-(1H-indol-3-yl)-1-[4-(toluene-4-sulfonyl)-piperazine-1-carbonyl]-ethyl}-amide;

7-Amino-heptanoic acid [1{4-[4,4-bis-(4-fluoro-phenyl)-butyl]-piperazine-1-carbonyl}-2-(1H-indol-3-yl)-ethyl]-amide;

8-Amino-octanoic acid {1-(1H-indol-3-ylmethyl)-2-oxo-2-[4-(2-oxo-2,3-dihydro-benzoimidazol-1-yl)-piperidin-1-yl]-ethyl}-amide;

6-Amino-hexanoic acid {1-(1H-indol-3-ylmethyl)-2-oxo-2-[4-(2-oxo-2,3-dihydro-benzoimidazol-1-yl)-piperidin-1-yl]-ethyl}-amide; and

7-Amino-heptanoic acid [2-(1H-indol-3-yl)-1-(2-p-tolyl-ethylcarbamoyl)-ethyl]-amide.

Additional compounds of the invention include:

7-Amino-heptanoic acid {2-(1H-indol-3-yl)-1-[2-(1H-indol-3-yl)-ethylcarbamoyl]-propyl}-amide;

7-Amino-heptanoic acid {1-[2-(1H-indol-3-yl)-ethylcarbamoyl]-2-naphthalen-1-yl-ethyl}-amide;

7-Amino-heptanoic acid {1-[2-(1H-indol-3-yl)-ethylcarbamoyl]-2-naphthalen-2-yl-ethyl}-amide;

7-Amino-heptanoic acid {1-[2-(1H-indol-3-yl)-ethylcarbamoyl]-2-pheny.-ethyl}-amide;

7-Amino-heptanoic acid {1-[2-(1H-indol-3-yl)-ethylcarbamoyl]-2-pyridine-2-yl-ethyl}-amide;

7-Amino-heptanoic acid {1-[2-(1H-indol-3-yl)-ethylcarbamoyl]-3-phenyl-propyl}-amide;

7-Amino-heptanoic acid [1-(2-benzofuran-3-yl-ethylcarbamoyl)-2-(1H-indol-3-yl)-ethyl]-amide;

7-Amino-heptanoic acid [1-[3-(2-hydroxy-phenyl)-propylcarbamoyl]-2-(1H-indol-3-yl)-ethyl]-amide;

7-Amino-heptanoic acid [1-(indan-2-ylcarbamoyl)-2-(1H-indol-3-yl)-propyl]-amide;

7-Amino-heptanoic acid [1-(indan-2-yl-methyl-carbamoyl)-2-(1H-indol-3-yl)-propyl]-amide; and

7-Dimethylamino-heptanoic acid [1-{[2-(5-fluoro-1H-indol-3-yl)-ethyl]-methyl-carbamoyl}-2-(1H-indol-3-yl)-ethyl]-amide.

The compounds and pharmaceutical compositions of this invention include all conformational isomers of compounds of formula I (e.g., cis and trans isomers, whether or not involving double bonds). The compounds of the invention include all optical isomers of the compounds of formula I (e.g., enantiomers and diastereomers), as well as racemic, diastereomeric and other mixtures of all such isomers. This invention further relates to tautomers and stereoisomers of the compounds of formula (I), and mixtures of any of the aforementioned forms. As will be described below in greater detail, certain isomeric structures are preferred.

The present invention also relates to the pharmaceutically acceptable acid addition salts of compounds of the formula (I). The acids which are used to prepare the pharmaceutically acceptable acid addition salts of the aforementioned base compounds of this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3- naphthoate)]salts.

With respect to the relatively limited number of compounds that so permit, the invention also relates to base addition salts of formula (I). The chemical bases that may be used as reagents to prepare pharmaceutically acceptable base salts of those compounds of formula I that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

The subject invention also includes isotopically-labelled compounds, which are identical to those recited in Formula (I), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of Formula (I) of this invention and prodrugs thereof can generally be prepared by carrying out the procedures disclosed in the Schemes and/or in the Examples and Preparations below, by substituting a readily available isotopically labelled reagent for a non-isotopically labelled reagent.

The present invention also relates to a pharmaceutical composition for increasing growth hormone secretion in a mammal, including a human, comprising an effective amount of a compound according to formula 1, and a pharmaceutical carrier. The present invention also relates to a pharmaceutical composition for increasing gastrin secretion or glucagon secretion in a mammal, comprising an effective amount of a compound according to formula 1, and a pharmaceutical carrier.

The present invention also relates to a pharmaceutical composition for the treatment of diseases characterized by decreased levels of growth hormone, glucagon, or gastrin in a mammal, including a human, comprising an amount of a compound of formula (I) effective in such treatments and a pharmaceutically acceptable carrier. The present invention also relates to a pharmaceutical composition for the treatment of diseases in a mammal, including a human, wherein treatment can be effected by inhibiting the binding of somatostatin to the sst2-type receptor therefor, comprising an effective amount of a compound according to formula 1, and a pharmaceutical carrier.

The present invention relates to a method for treating growth hormone deficiency in a mammal, including a human. The present invention also relates to elevating the level of growth hormone in a mammal, including a human, wherein this is beneficial to the mammal nothwithstanding that the natural levels of growth hormone present in the mammal are within the normal range. In the practice of said method, there is administered a pharmaceutical composition of the invention comprising a compound according to formula (1), and a pharmceutical carrier.

Similarly, the methods of the invention provide for increasing gastrin secretion or glucagon secretion in a mammmal, including a human, where this is medically appropriate. For example, gastrin is involved in protection of gastric mucosa against damage by chemical substances, e.g. alcohol (S. J. Konturek et al., European Journal of Pharmacology, 278(3), pp. 203-212, 1995). Glucagon is a counter-regulatory hormone that is used to treat hypoglycemia, and causes positive inotropic and chronotropic effects without the need for beta-i adrenoceptor stimulation. It also can be used to correct beta-blocker, verapamil and imipramine overdose, and is used as adjunctive therapy in shock situations, for heart failure, and in treating postcountershock asystole (see C.M. White, Journal of Clinical Pharmacology,. 39(5), pp. 442-447, 1999)

In preferred examples of the invention, there are provided methods for treating a human for one or more symptoms of insufficient growth hormone secretion, or one or more conditions that may occur therewith and be exacerbated thereby, wherein said condition is selected from frailty, hypoglycemia, wrinkled skin, slow skeletal growth, reduced immune function, reduced organ functon, fertility disorders, bone disease, AIDS-related complex, cachexia, cardiac failure, ischemic heart disease, colon disease, metabolic disorders, renal failure, muscular dystrophy, and Turners syndrome, comprising administering an effective amount of a pharmaceutical composition as aforementioned. It will be appreciated that numerous of the above conditions also affect non-human mammals, and treatment of such conditions is also within the practice of the invention.

In a further preferred example of the invention, there is provided a method for treating a non-human mammal to enhance the growth and performance thereof, comprising administering an effective amount of a pharmaceutical composition as aforementioned. Enhancement of growth and performance includes, for example, increased feed efficiency, improved milk yield or fertility, and increased leanness.

A highly preferred example of the invention provides a method whereinby secretion of growth hormone, gastrin, or glucagon can be increased on a sustained basis in a mammal, including a human, in need thereof, comprising adminstering a dose of a pharmaceutical composition as aforementioned. According to this example of the invention, physiologically adverse consequences of artificial fluctuations in the circulating (or locally needed) concentrations of these hormones can be avoided.

Although the pharmaceutical compositions and methods of the invention are described primarily in terms of use with humans, and non-human mammals, the skilled practitioner will immediately appreciate that the invention, in many of its aspects, may be usefully practiced with respect to birds, such as chickens and turkeys, and also fishes.

### Definitions

In connection with the practice of the invention, the following definitions will generally apply.

The term “treating”, as used herein, refers to reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

The term “alkyl”, as used herein, unless otherwise indicated, includes saturated monovalent hydrocarbon radicals having straight, branched or cyclic moieties or combinations thereof. Similarly, the terms “alkenyl” and “alknyl” define hydrocarbon radicals having straight, branched or cyclic moities wherein at least one double bond, or at least one triple bond, respectively, is present. Such definitions also apply when the alkyl, alkenyl or alkynyl group is present within another group, such as alkoxy or alkylamine.

The term “alkoxy”, as used herein, includes O-alkyl groups wherein “alkyl” is as defined above.

The term “halo”, as used herein, unless otherwise indicated, includes fluoro, chloro, bromo or iodo.

An “aryl” group as used herein, unless otherwise indicated, includes an organic radical derived from a monocyclic or bicylic (C₆-C₁₀) aromatic hydrocarbon compound by removal of a hydrogen radical from a ring carbon of the aryl compound. An aryl group is optionally substituted by one or more substituents wherein, unless otherwise indicated, selection of each optional substituent is independent of selection of any other optional substituents, and perferably the number of optional substituents is between 0 and 3, more preferably between 0 and 2. It will be appreciated that the preferred number of substituents is determined in part by facility of synthesis. Representative aryl groups are phenyl and naphthyl.

A “heteroaryl” group as used herein, unless otherwise indicated, includes an organic radical derived from a monocyclic or bicyclic (C₁-C₉) aromatic heterocyclic compound by removal of a hydrogen radical from a ring atom of the heteroaryl compound, said ring atom being uncharged in said compound. A heteroaryl group is optionally substituted by one or more substituents wherein, unless otherwise indicated, selection of each optional substituent is independent of selection of any other optional substituents, and perferably the number of optional substituents is between 0 and 3, more preferably between 0 and 2. It will be appreciated that the preferred number of substituents is determined in part by facility of synthesis. Representative heteroaryl groups include furyl-, thienyl-, thiazolyl- pyrazolyl-, isothiazolyl-, oxazolyl-, isoxazolyl-, pyrrolyl-, triazolyl-, tetrazolyl-, imidazolyl-, 1,3,5-oxadiazolyl-, 1,2,4-oxadiazolyl-, 1,2,3-oxadiazolyl-, 1,3,5-thiadiazolyl-, 1,2,3-thiadiazolyl-, 1,2,4-thiadiazolyl-, pyridyl-, pyrimidyl-, pyrazinyl-, pyridazinyl-, 1,2,4-triazinyl- 1,2,3-triazinyl-, 1,3,5-triazinyl-, pyrazolo[3,4-b]pyridinyl-, cinnolinyl-, pteridinyl-, purinyl-, 6,7-dihydro-5H-[1]pyrindinyl-, benzo[b]thiophenyl-, 5, 6, 7, 8-tetrahydro-quinolin-3-yl-, benzoxazolyl-, benzothiazolyl-, benzisothiazolyl-, benzisoxazolyl-, benzimidazolyl-, thianaphthenyl-, isothianaphthenyl-, benzofuranyl-, isobenzofuranyl-, isoindolyl-, indolyl-, indolizinyl-, indazolyl-isoquinolyl-, quinolyl-, phthalazinyl-, quinoxalinyl-, quinazolinyl-, benzoxazinyl-; and the like.

A “cycloalkyl” group as used herein, unless otherwise indicated, includes an organic radical derived from a monocyclic (C₃-C₁₀)cycloalkyl compound, by removal of a hydrogen radical from a ring carbon of the cycloalkyl compound. A cycloalkyl group is optionally substituted by one or more substituents wherein, unless otherwise indicated, selection of each optional substituent is independent of selection of any other optional substituents, and perferably the number of optional substituents is between 0 and 3, more preferably between 0 and 2. It will be appreciated that the preferred number of substituents is determined in part by facility of synthesis. Representative cycloalkyl groups include cyclopropyl, cyclobutyl, cyclopentyl, cyclohexyl, cycloheptyl, cyclopropenyl, cyclobutenyl, cyclopentenyl, cyclohexenyl, cycloheptenyl, 1,3-cyclobutadienyl, 1,3-cyclopentadienyl, 1,3-cyclohexadienyl, 1,4-cyclohexadienyl, 1,3-cycloheptadienyl, 1,4-cycloheptadienyl, 1,3,5-cycloheptatrienyl, bicyclo[3.2.1]octane, bicyclo [2.2.1] heptane, and the norborn-2-ene unsaturated form thereof. Thus, the term cycloalkyl also includes cycloalkenyl groups having one or two double bonds.

A “heterocycloalkyl” group as used herein, unless otherwise indicated, includes an organic radical derived from a monocyclic (C₃-C₁₀)heterocycloalkyl compound by removal of a hydrogen radical from a ring atom of the heterocycloalkyl compound. A heterocycloalkyl group is optionally substituted by one or more substituents wherein, unless otherwise indicated, selection of each optional substituent is independent of selection of any other optional substituents, and perferably the number of optional substituents is between 0 and 3, more preferably between 0 and 2. It will be appreciated that the preferred number of substituents is determined in part by facility of synthesis. Representative heterocycloalkyl groups include pyrrolidinyl-, tetrahydrofuranyl-, dihydrofuranyl-, tetrahydropyranyl-, pyranyl-, thiopyranyl-, aziridinyl-, oxiranyl-, methylenedioxyl-, chromenyl-, isoxazolidinyl-, 1,3-oxazolidin-3-yl-, isothiazolidinyl-, 1,3-thiazolidin-3-yl, 1,2-pyrazolidin-2-yl, 1,3-pyrazolidin-1-yl, piperidinyl-, thiomorpholinyl-, 1,2-tetrahydrothiazin-2-yl, 1,3-tetrahydrothiazin-3-yl, tetrahydrothiadiazinyl-, morpholinyl-, 1,2-tetrahydrodiazin-2-yl, 1,3-tetrahydrodiazin-1-yl, tetrahydroazepinyl, piperazinyl-, and chromanyl-.

In connection with the terms “aryl” group, “heteroaryl” group, “cycloalkyl” group and “heterocycloalkyl” group, as herein defined, the term “optionally substituted” means that one or more chemically and pharmaceutically acceptable functional groups may be bonded thereto. Such a group contributes properties useful to production, storage, or use of the inventive compounds as pharmaceuticals, or at least does not substantially negate their pharmacological activity. Such suitable substituents may be determined by those skilled in the art. Illustrative examples of suitable substituents include, but are not limited to,. hydroxy, halo, amino, trifluoromethyl, carboxy, (C₁-C₆)alkoxy-, (C₁-C₆)acyloxy-, (C₁-C ₆)alkylamino-, ((C₁-C₆)alkyl)₂amino-, (C₁-C₆)acylamino-, cyano, nitro, (C₁-C₆)alkyl-, (C₂-C₆)alkenyl-, (C₂-C₆)alkynyl-, (C₁-C₆)acylamino-, cyano(C₁-C₆)alkyl-, trifluoromethyl(C₁-C ₆)alkyl-, nitro(C₁-C₆)alkyl-, (C₁-C₃)alkyl(difluoromethylene)(C ₁-C₃)alkyl-, (C₁-C₆)acylamino(C₁-C₆)alkyl-, (C,-C₆)alkoxy(C₁-C₆)acylamino-, amino(C₁-C₆)acyl-, amino(C₁-C₆)acyl(C₁-C₆)alkyl-, (C₁-C6)alkylamino(C₁-C₆)acyl-, ((C₁-C₆)alkyl)₂amino(C₁-C₆)acyl-, (C₃-C₁₀)cycloalkyl(C₁-C₆)alkyl-, (C₁-C₆)acyloxy(C₁-C₆)alkyl-, (C₂-C₆)alkoxy(C₁-C ₆)alkyl-, piperazinyl(C₁-C₆)alkyl-, (C₁-C₆)acylamino(C₁-C ₆)alkyl-, (C₆-C₁₀)aryl(C₁-C₆)alkoxy(C₁-C6)alkyl-, (C ₁-C₉)heteroaryl(C₁-C₆)alkoxy(C₁-C₆)alkyl-, (C₁-C₆)alkylthio(C₁-C₆)alkyl-, (C₆-C₁₀)arylthio(C₁-C₆)alkyl-, (C ₁-C₆)alkylsulfinyl(C₁-C₆)alkyl- (C₆-C₁₀)arylsulfinyl(C₁-C₆)alkyl-, (C₁-C₆)alkylsulfonyl(C₁-C₆)alkyl-, (C₆-C₁₀)arylsulfonyl(C₁-C₆)alkyl-, amino(C₁-C₆)alkyl-, (C₁-C₆)alkylamino(C₁-C₆)alkyl-, (C₁-C₆)alkyl(difluoromethylene)-, (C₁-C₃)alkyl(difluoromethylene)(C₁-C₃)alkyl-, (C₁-C₆)alkoxy(C₁-C₆)acyl-, (C₁-C₆)alkylamino(C₁-C₆)acyl-, ((C₁-C₆)alkyl)₂amino(C₁-C₆)acyl-, (C₆-C ₁₀)aryl-, (C₁-C₉)heteroaryl-, (C₆-C₁₀)aryl(C₁-C₆)alkyl-, (C₁-C₉)heteroaryl(C₁-C₆)alkyl-, (C₆-C₁₀)aryl (C₆-C₁₀)aryl-, (C₆-C₁₀)aryl(C₆-C₁₀)aryl(C₁-C₆)alkyl- (C₃-C₁₀)cycloalkyl-, (C₃-C₆)cycloalkyl(C₁-C₆)alkyl-, (C₃-₁₀)heterocycloalkyl-, (C₃-C₁₀)heterocycloalkyl(C₁-C₆)alkyl-, hydroxy (C₂-C₆)alkyl-, (C₁-C₆)acyloxy(C₂-C₆)alkyl-, (C₁-C₆)alkoxy(C₂-C₆)alkyl-, piperazinyl(C₁-C₆)alkyl-, (C₁-C₆)acylamino(C₁-C₆)alkyl-, (C₆-C₁₀)aryl(C₁-C₆)alkoxy(C₁-C₆)alkyl-, (C₁-C₉)heteroaryl(C₁-C₆)alkoxy( C₁-C₆)alkyl-, (C₁-C₆)alkylthio(C₁-C₆)alkyl-, (C₆-C₁₀)arylthio(C₁-C₆)alkyl-, (C₁-C₆)alkylsulfinyl(C₁-C₆)alkyl-, (C₆-C₁₀)arylsulfinyl(C₁-C₆)alkyl-, (C₁-C₆)alkylsulfonyl(C₁-C₆)alkyl-, (C₆-C₁₀)arylsulfonyl(C₁-C₆)alkyl-, amino(C₁-C₆)alkyl-, (C₁-C₆)alkylamino(C₁-C₆)alkyl-, and ((C₁-C₆)alkyl) ₂amino(C₁-C₆)alkyl.

Further aspects of the invention are described in accord with the Detailed Description of the Invention which follows directly.

### DETAILED DESCRIPTION OF THE INVENTION

According to the practice of the present invention, the secretion of growth hormone (GH) from cells (such as those of the anterior pituitary) is facilitated by inhibiting the somatostatin-induced (and G-protein coupled) mechanisms that otherwise naturally act to oppose said secretion. Without being limited as to theory, these somatostatin-induced mechanisms act to oppose both calcium ion and cyclic AMP-mediated signals that otherwise enhance fusion with the cell membrane of cytoplasmic granule structures that contain growth hormone, and thus the subsequent release (secretion) of GH.

The present invention provides an effective approach to the treatment of frailty in older persons, which may be caused, in whole or part, by insufficient levels of growth hormone (GH), or impairment of any of several downstream physiological effects normally associated with growth hormone secretion.

It is generally recognized that GH is important to the maintenance of connective and muscle tissue in adults, and may help, to some extent, to increase muscle mass. Thus growth hormone may be used to assist elderly patients even when growth hormone levels per se are not the cause of, for example, weakness, or attrition of muscle and connective tissues.

The practice of the invention benefits other patients, such as children, when it can be demonstrated that secretion of GH is inadequate, but is subject to enhancement. Deficiency in GH secretion, or resultant GH activity, may arise in several ways. For example, the gene sequence that encodes GH may be expressed in the nucleus at subnormal levels, processing of resultant RNA transcript or nascent polypeptide may be defective, or fusion of cytoplasmic GH storage granules with the cell membrane (with resultant release of GH) may be defective. Additionally, the patient may possess an allele of the GH gene that encodes a mutant protein having less biological activity. Alternatively, there may be an underlying deficiency of GHRH, or a defect in the GHRH receptor, or defects in the the GHRP receptor or deficiency of its endogenous ligand, or in respective signalling mechanisms. Additionally, there may be an excess of somatostatin. In all such cases, the resultant physiological deficiency can be treated by administration of the pharmaceutical compounds of the invention.

In a further aspect of the invention, the performance and growth rate of non-human mammals, such as livestock, is enhanced by appropriate administration of the compounds disclosed herein. Additionally, companion animals, and particularly older companion animals also benefit upon administration of the present compounds.

Under appropriate circumstances, somatostatin antagonists may also exhibit the properties of agonists, and are thus recognized as useful therapeutics in the treamtment of diabetes, for example, see H. Grønbæck et al., Prog. Basic Clin Pharmacol. (Basel), 10, pp. 103-128, 1996. Somatostatin agonists are also recognized (see WO 98/44922) as useful therapeutics in the treatment of, for example, diabetic retinopathy, acromegaly, rheumatoid arthritis, neuropathic and visceral pain, irritable bowel syndrome, Crohn's disease, and are useful to inhibit cell proliferation associated with cancer, and to prevent restenosis following angioplasty.

Additionally, sst2 ligands can evidence affinity for other G protein-coupled receptors including the melanocortin receptor, the MCH receptor, and MCR4. It is also expected that sst2 ligands will evidence affinity for the MCH receptor SLC1 (somatostatin-like receptor 1) since it is more than 50% homologous to sst2. Accordingly, the compounds of the present invention are also useful in the treatment of medical conditions mediated through these receptors including, for example, treatment or prevention of obesity, diabetes mellitus, erectile disfuinction and female sexual disfunction. Additionally, the compounds of the present invention are useful to modulate appetite and metabolic rate. In particular, the compounds of the present invention are useful to stimulate the appetite of mammals for the trearment of diseases/disorders associated with inappropriate food intake and weight loss, and for example, to enhance growth and survivability of neonates in livestock.

Although the compounds of the present invention act to indirectly facilitate release of mature growth hormone from the cytoplasmic storage granules of cells, additional therapeutic substances are known that can directly enhance such secretion, and further, can indirectly enhance production of growth hormone by via enhanced expression of GH-encoding DNA in the cell nucleus. In this regard, both growth hormone releasing peptide (GHRP) and growth hormone releasing hormone (also known as growth hormone releasing factor, GHRH/GRF) which act to release GH from cytoplasmic storage granules have been mentioned. Since the release of GH from such granules has been implicated as a signal triggering production of additional GH protein in the cells, it is expected that GH levels may be properly maintained in patients using a “push-pull” approach.

Accordingly, a further preferred example of the invention provides for the co-administration of the somatostain-antagonist compounds of the present invention and GHRP or GHRH, or other substances of like effects. Medical treatment with GHRP (or GHRH) alone is described in the following representative publications: M. Thorner et al., Journal Of Clinical Endocrinology And Metabolism, 81(3), pp. 1189-1196, 1996; S. G. Celia et al., Peptides, 16(1), pp. 81-86, 1995; M. A. Bach et al., Journal Of The American Geriatrics Society, 44(9), S10, 1996; and J. A. Aloi et al., Journal Of Clinical Endocrinology And Metabolism, 79(4), pp. 943-949, 1994.

Further, since growth hormone is very labile, and its half-life in the body is very short, it is difficult to provide a safe dosing program for direct administration of growth hormone itself, which avoids wide swings in circulating levels of the hormone. Current sustained release technologies for direct administration of growth hormone can be improved upon. In this regard, the practice of the present invention is particularly valuable to the clinician, since by only indirectly raising GH levels, the hormone's release profile remains, at least in part, under the control of the body's own regulatory feedback systems, and fluctuations in the levels of circulating GH are damped over time. Additionally, the compounds of the present invention may themselves be administered by sustained release mechanisms. It is also recognized that patients sometimes inadvertently skip doses, and various technologies exist to provide continuous dosing via the digestive tract including, for example, osmotic systems. In this regard, the pharmaceutical compositons of the invention are preferably administered according to the technology disclosed in U.S. Pat. 4,612,008.

In the preferred practice of the invention, compounds show selectivity for the sst2 receptor compared with other receptor subtypes, for example sst1, sst3, sst4 and sst5. This selectivity minimizes the chance that other molecular biological or biochemical pathways will be adversely affected while growth hormone secretion is being upregulated. Most preferably, the affinity of a compound for the sst2 type receptor should be at least about 10 times greater than for receptors of the other sst-subtypes.

It should be noted that the compounds of the invention may work by more than one mechanism, including those unrelated to interaction at an sst-type receptor, and the utility of the present compounds in the practice of the invention, including for use in treating other disease states not particularly mentioned herein, is not limited by any particular theory as desrcibed herein or by those theories that is generally recognized by those skilled in the art.

Additionally, the compounds of the present invention may interact beneficially with sst-type receptors other than sst2, and may provide therapeutic benefits by acting as somatostatin agonists, rather than antagonists, at sst2 or other sst-type receptors.

As aforementioned. the compounds of this invention include all conformational isomers (eq., cis and trans isomers, whether or not involving double bonds), tautomers, and all optical isomers of compounds of the formula I (e.g., enantiomers and diastereomers), as well as racemic, diastereomeric and other mixtures of all such isomers.

Additionally, many of the groups of the present compounds may be optionally substituted. As aforementioned, such substituents contribute properties useful to production, storage, or use of the inventive compounds as pharmaceuticals, or at least does not substantially negate their pharmacological activity. It will be appreciated that selection of optional substituents is further guided by principles recognized in the art, and/or is capable of validation through the use of the assays described in the present specification.

### Pharmaceutical formulations

The compounds of the present invention that are basic in nature are capable of forming a wide variety of different salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared, for example, by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of the present invention that are acidic in nature, are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

In a preferred example of the invention, the compounds of the present invention may be formulated with additional pharmaceutically active substances that directly or indirectly (1) facilitate production and storage in cells of additional growth hormone, or precursor polypeptides thereof, or (2) facilitate release of GH. Such additional substances include growth hormone releasing peptide (GHRP), growth hormone releasing hormone (GHRH), pituitary adenylate cyclase activating polypeptide (PACAP), dopaminergic agonists (e.g. bromocriptine), beta-adrenergic agonists (e.g. isoproterenol) and alpha 1-adrenergic agonists (e.g. methoxamine). For background information see E. O Soyoola et al., Proceedings of the Society for Experimental Biology & Medicine, 207(1), pp. 26-33, 1994; V. Locatelli et al., Pediatric Research, 36(2), pp. 169-74, 1994; and B. Velkeniers et al., Journal of Endocrinology, 143(1), pp. 1-11, 1994.

Equivalently, the additional pharmaceutically active substances may be provided as a separate formulation which is co-administered, or administered at some other timepoint(s) in the course of treatment.

This invention also encompasses pharmaceutical compositions containing prodrugs of compounds of the formula I. This invention also encompasses methods of treating or preventing disorders that can be treated or prevented by decreasing the levels of somatostatin comprising administering prodrugs of compounds of the formula I. Compounds of formula I having free amino, amido, hydroxy or carboxylic groups can be converted into prodrugs. Prodrugs include compounds wherein an amino acid residue, or a polypeptide chain of two or more (e.g., two, three or four) amino acid residues which are covalently joined through peptide bonds to free amino, hydroxy or carboxylic acid groups of compounds of formula I. The amino acid residues include the 20 naturally occurring amino acids commonly designated by three letter symbols and also include, 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of formula I through the carbonyl carbon prodrug sidechain.

One of ordinary skill in the art will also appreciate that when using the compounds of the invention in the treatment of a specific disease, that the compounds of the invention may be combined with various existing therapeutic agents used for that disease, or for other metabolically related or unrelated disease states that may occur simultaneously. As aforementioned, the additional pharmaceutically active substances may be provided as a separate formulation which is co-administered, or administered at some other timepoint(s) in the course of treatment.

The compounds of the invention can also be used in combination with existing therapeutic agents such as the above-mentioned growth hormone secretagogues for the treatment of growth hormone deficiency.

For the treatment of growth hormone deficiency, the compounds of the invention may be combined with agents such as recombinant growth hormone which is marketed by Genentech and licensees (Neutropin, Genotropin and Protropin), Bio-Technology General and licensees (Zomacton, Growject, Elvetium and SciTropin), Novo Nordisk (Norditropin), LG Chem (Eutropin), Ares Serono (Saizen and Serostim), Eli Lilly Co (Humatrope), Monsanto (Posilac brand of bovine growth hormone) and Alpharma (Reporcin brand of swine growth hormone).

The compounds of the invention can also be used in combination with existing therapeutic agents such as Geref (sermorelin, GHRH) from Serono Laboratories Inc.

The compounds of the invention can also be used in combination with existing therapeutic agents such as anabolic steroids, e.g. androisoxazol androstanolone (DHT, dihydrotestosterone, Stanolone, Anabolex, Andractrim), bolandiol, bolasterone, bolazin, boldenone (Equipoise), calusterone, clostebol (chlortestosterone, Steranabol, Alfa Trofodermin, Dermanabol, Trofodermin, Trofoseptine), danazol (Cyclomen, Danocrine), dehydrochlormethyltestosterone (turinabol, Oral-turinabol), drostanolone (dromostanolone, Drolban, Masterid, Masteril, Masteron, Metormon, Premastril), estradiol, ethylestrenol, fluoxymesterone (Halotestin, Ora-Testryl, Android-F), formebolone, furazabol (Miotolon), mestanolone, mesterolone (Proviron, Pluriviron), methandienone (methandrostenolone, Metaboline), methandriol, methenolone (Primobolan), methyltestosterone (Methandren, Premarin with methyltestosterone, Android, Oreton, Testred, Methyltestosterone tabs, Geri-Bons, Geri-tabs, Dermonal), mibolerone (Cheque), nandrolone (Deca-Durabolin, Durabolin, Nandrabolin, Anabolin, Androlone, Hybolin, Nandrobolic), norclostebol, norethandrolone (Nilevar), oxabolone, oxandrolone (Anavar), oxymesterone (Oranabol), oxymetholone (Anapolon 50, Androyd, Anadrol, Anasteron, Dynasten, Oxitosona, Plenastril, Synasteron, Zenalosyn), penmesterol, prasterone, quinbolone, stanozolol (Winstrol, Winstrol-V, Stromba, Strombaject), stenbolone, testosterone (Malogen, Delatestryl, Malogen, Neo-pause, PMS-testosterone Enanthate, Andriol, Duogex, Neo-Pause, Climacteron, Orchisterone-P, Oreton, Anadiol, Anatest, Testos-100, Heifer-aid, Synovex-H), tibolone, trenbolone (Parabolan, Finaject) or zeranol.

The compounds of the invention can also be used in combination with existing therapeutic agents such as Somazon (mecasermin, recombinant insulin-like growth factor 1) from Fujisawa.

For the treatment of older patients with osteoporosis, suitable agents to be used in combination with the compounds of the invention include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib and rofecoxib, analgesics and intraarticular therapies such as corticosteroids and hyaluronic acids such as hyalgan and synvisc.

The compounds of the present invention may also be used in combination with osteoporosis agents such as lasofoxifene, raloxifene, droloxifene or fosomax and immunosuppressant agents such as FK-506 and rapamycin.

The compounds of the present invention may also be used in combination with immunostimulant agents for the treatment of reduced immune function.

The compounds of the present invention may also be used in combination with fertility agents such as human menopausal gonadotropin, chorionic gonadotropin, follicle stimulating hormone, nafarelin, triptorelin, cetrorelix, and ganirelix for the treatment of infertility.

The compounds of the present invention may also be used in combination with AIDS therapies for the treatment of AIDS-related complex.

The compounds of the present invention may also be used in combination with anti-tumor necrosis factor agents such as infliximab (TNF monoclonal antibody) or etanercept (soluble TNF receptor) for the treatment of cachexia.

The compounds of the present invention may also be used in combination with potassium channel blockers, beta-blockers, anticoagulants or vasodilators for the treatment of heart disease.

The compounds of the present invention may also be used in combination with angiotensin II (ATII) antagonists or erythropoietin for the treatment of renal failure.

For administration to livestock, the compounds of the invention may also be used in combination with feed additives such as antibiotics (e.g. monensin, lasalocid, salinomycin, semduramicin, narasin, maduramicin, virginiamycin, polymixin, efrotomycin, avoparcin, lincomycin, bacitracin, bambermycins, novobiocin, erythromycin, oleandomycin, streptomycin, tylosin, penicillin, tetracycline, oxytetracycline, chlortetracycline, carbadox, olaquindox, neomycin, moenomycin, avilamycin, and flavophospholipol), repartitioning agents, beta-agonists ( e.g. Paylean, ractopamine, from Elanco), and also amiterol, bambuterol, bitolterol, broxaterol, buphenine, carbuterol, cimaterol, clenbuterol, clorprenaline, colterol, denopamine, dioxethedrine, dioxifedrine, dobutamine, dopexamine, doxaminol, etanterol, fenoterol, flerobuterol, formoterol, hexoprenaline, ibuterol, imoxiterol, isoetarine, isoxsuprine, levisoprenaline, mabuterol, mesuprine, metaterol, methoxyphenamine, nardeterol, orciprenaline, picumeterol, pirbuterol, prenalterol, procaterol, protokylol, quinprenaline, rimiterol, ritodrine, salbutamol, salmeterol, terbutaline, tretoquinol, tulobuterol, xamoterol and zilpaterol.

The compositions of the present invention may be formulated in a conventional manner using one or more pharmaceutically acceptable carriers. Thus, the active compounds of the invention may be formulated for oral, buccal, intranasal, parenteral (e., intravenous, intramuscular or subcutaneous) or rectal administration or in a form suitable for administration by inhalation or insufflation. The active compounds of the invention may also be formulated for sustained delivery.

For oral administration, the pharmaceutical compositions may take the form of, for example, tablets, chewable tablets, or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g.., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner, or blended with petfood or animal feed, or as a pre-mix for blending with animal feed.

The active compounds of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human is 0.1 to 100 mg of the active ingredient per unit dose which could be administered, for example, 1 to 4 times per day.

Aerosol formulations for treatment of the conditions referred to above in the average adult human are preferably arranged so that each metered dose or “puff” of aerosol contains 20 μg to 1000 μg of the compound of the invention. The overall daily dose with an aerosol will be within the range 0.1 mg to 100 mg. Administration may be several times daily, for example 2, 3, 4 or 8 times, giving for example, 1, 2 or 3 doses each time.

Injected doses are preferably administered from about once a month, up to about 1 to 4 times per day, at an individual dosing of 0.01-1 mg/kg (of active ingredient) and may be intramuscular, intravenous, or subcutaneous, for example.

As is well recognized, the precise dose, and method and timing of administration thereof, are capable of determination by those skilled in the art, and depend upon numerous factors including the activity of the therapeutic compound, the properties of the formulation thereof, the nature and location of the target tissue, and the particulars of the disease state as it exists in a particular patient. Additionally, when the compounds of the present invention are administered to a patient with additional pharmaceutically active substances, one or more pharmaceutical compositions may be used to deliver all of the active agents, which may be administered together, or at different times, as determined by those skilled in the pharmaceutical or medical arts.

The following reaction schemes illustrate preparation of compounds of the present invention. It will be appreciated that certain groups represented by subscripted letters (R₂, for example) in the Schemes do not always correspond with similarly defined component groups of the formula (I) compounds themselves, since certain functionalities of the reactants are modified, by definition, when the products are formed. R₁, R₂ and R₃ typically represent (C₁-C₆) alkyl groups, but can also be other groups such as (C₆-C₁₀)aryl or benzyl, for example, as provided for below

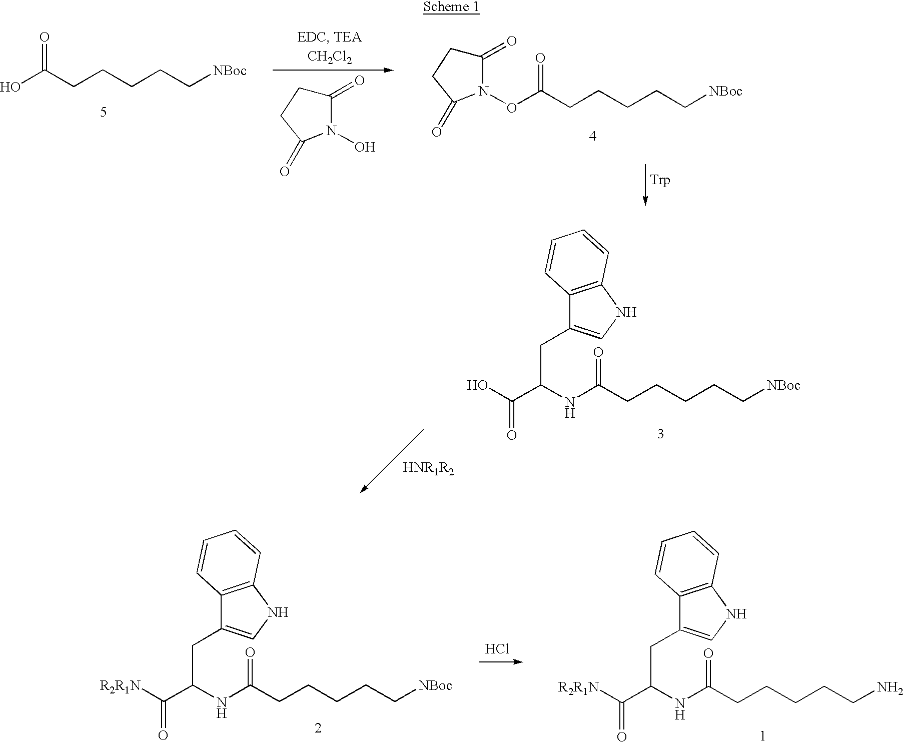

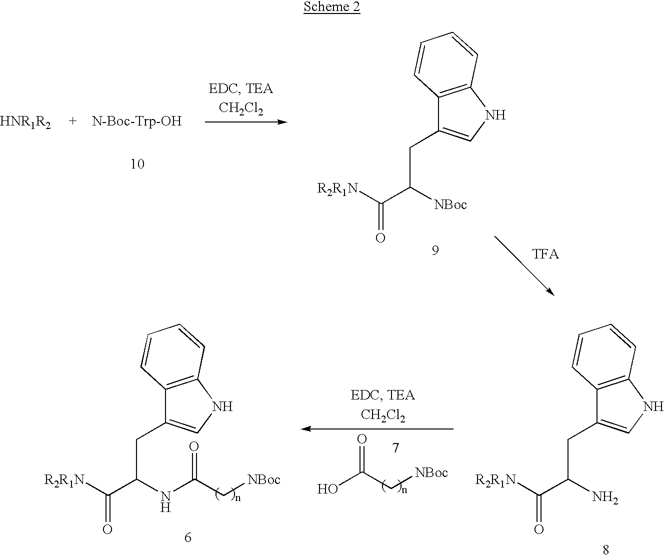

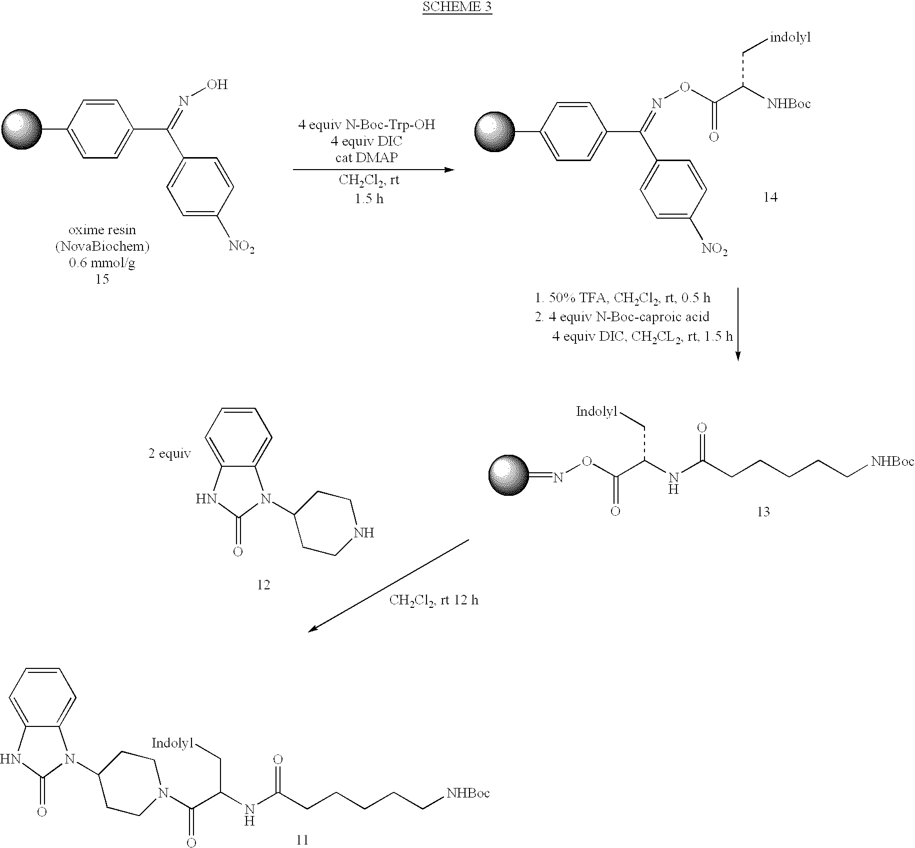

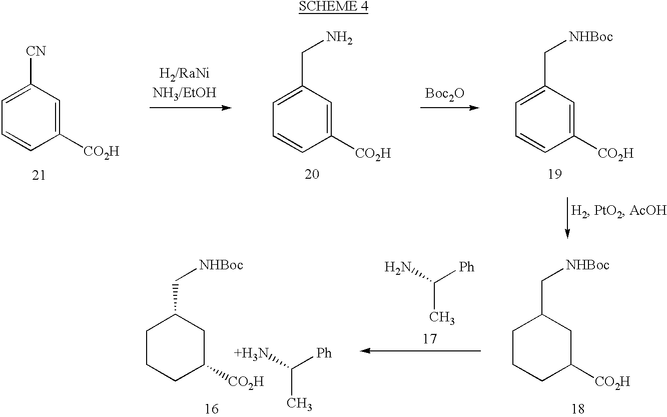

General reaction conditions

Generally speaking, the compounds of the present invention are made by a series of condensation reactions in which certain reactive groups are appropriately protected, and the sequence of condensation is controlled. Typically, alternative pathways exist to the same products, as the reactants may be coupled in more than one sequence. Generally speaking, the starting materials herein are commercially available or are readily prepared.

In Scheme 1, the group W component (here an ε-amino carboxylic acid) is first coupled to the group Z component (exemplified by tryptophan), after which a combined A-B moiety is added. According to the Scheme (see also Example 1), a Boc-protected ε-amino carboxylic acid (compound 5) is mixed with N-hydroxysuccinamide in a suitable solvent (such as methylene chloride, with TEA) followed by addition of EDC (1,3-dimethylaminopropyl-3-ethylcarbodiimide hydrochloride, 3.83 g, 20 mmol. The succinamide group of the resultant product, compound 4, is readily displaced by tryptophan under appropriate conditions (for example in the presence of triethylamine in a solvent of 4:1 dioxane:water, followed by dilution with water and acidification to pH 3) to yield compound 3.

According to this Scheme, the groups defined by A-B according to general formula 1 are next added (3→2) using “general procedure A” as outlined, for example, in Example 1, step 3 below. In a typical case, condensation using EDC is employed in a suitable environment of triethylamine and methylene chloride. Broadly speaking, the reagent HNR₁R₂ is a primary or secondary amine, whether alipahtic or aromatic. By appropriate selection of R₁ and R₂ groups, a wide variety of A-B moieties can be added, through reaction of the amine nitrogen of HNR₁R₂ at the free carboxylic acid group of compounds 3.

For example, appropriate compounds HNR₁R₂ include A—(CH₂)ₖ—NH₂ such as

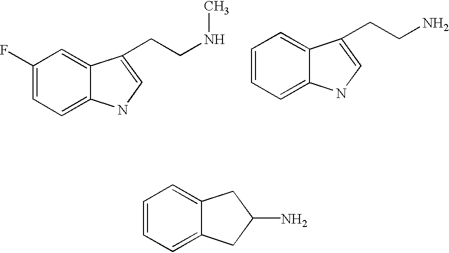

Similarly, the HNR₁R₂ reactant may provide the A-B component of the compounds of

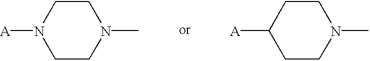

the invention as

Under similar reaction conditions, aromatic and aliphatic alcohols are also suitable for use as reagents HNR₁R₂. Thus the A-B moiety of the compounds of the invention can be delivered, for example, as A—OH or A—(CH₂)ₖ—OH.

In Scheme 1, final product compounds 1 can be generated under acidic conditions by hydroylsis of the N-terminal Boc group.

Scheme 2 provides an equivalent pathway to similar products. In this Scheme, reagent HNR₁R₂ is reacted with compound 10 (Boc-protected group Z, illustrated herein by tryptophan) to yield compound 9. Conditions for this reaction are similar to those used in Scheme 1, that is, using EDC in a solution of triethylamine (TEA) and methylene chloride. The Boc group is then removed from compound 9 to form compound 8 under acidic conditions (for example using trifluoroacetic acid). Component W is next provided via reagent 7, exemplified here as a Boc-protected aminocarboxylic acid, with the reaction again conducted using EDC in a solvent environment of TEA and methylene chloride. The resultant compound 6 may be converted into a final product compound by hydrolysis of the remaining Boc group, again under acidic conditions.

Scheme 3 provides a further pathway wherein an oxime resin (NovaBiochem) is used to localize the intermediate compounds as the reaction sequence proceeds. According to this Scheme, oxime resin 15 is reacted with a compound that provides the Z component (as illustrated, N-Boc protected tryptophan), thus localizing the Z component on the resin (structure 14). The reaction is conducted, for example, in a solvent of methylene chloride, using diisopropylcarbodiimide (DIC) and a catalytic amount (typically 5 mole percent) of dimethylaminopyridine (DMAP).

Subsequently, structure 14 is subjected to acidic conditions, for example, 50% TFA in methlyene chloride for 30 minutes at room temperature, in order to deprotect the α-amino group of the structure's tryptophan moiety. 7-tert-butoxycarbonylaminoheptanoic acid is then added in the presence of an equal amount of DIC in methlyene chloride to generate structure 13.

Compound 12 (which provides moieties A-B of the final product, and is equivalent to HNR₁R₂ in Scheme 1) is then reacted with structure 13 thereby displacing the resin, and generating product 11. Appropriate conditions include use of a solvent of methylene chloride, for a suitable incubation period such as for 12 hours at room temperature. The range of reactants A-B represented by structure 12 is essentially the same as that provided by HNR₁R₂ in Scheme 1 (alcohols or primary or secondary amines, except that here aromatic amines are likely to have insufficient nucleophilicity). Final product can be generated from compounds 11 by acid hydroylsis of the Boc group protecting the aminoheptanoic acid. Suitable environments include use of HCl or TFA.

Scheme 4 relates to circumstances where the Boc-protected aminocarboxylic acid (see Scheme 1, compound 5; Scheme 2, compound 7; and Scheme 3, 2nd reaction performed on structure 14) provides a group W according to alternative (b).

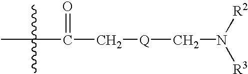

and in particular, outline representative syntheses of component W wherein Q is, for example, cyclohexane or pyridine. Thus, Scheme 4 permit synthesis of compounds similar to compounds of Schemes 1-3, but wherein the representative carboxylic acid moiety “W” thereof is replaced by a moiety that includes a cyclohexane group, for example.

Referring to Scheme 4, compounds 16, having the stereospecificity indicated in Scheme 4, are prepared from racemic compounds 18 by chiral resolution with stereospecific α-methylbenzylamine 17, followed by selective purification, such as by crystallization.

Compounds 18 may be prepared from the corresponding aromatic compounds 19 by reduction with hydrogen, for example, under appropriate conditions. Compounds 19 in turn are prepared from the corresponding (unprotected) compounds 20 by reaction with BOC anhydride under standard conditions. Finally, compounds 20 may be prepared from available starting materials 21, by reduction of the cyano group with hydrogen over a Raney nickel preparation.

The following are representative compounds of the invention.

### Example 1

7-Amino-heptanoic acid {1-(1H-indol-3-ylmethyl)-2-oxo-2-[4-(2-oxo-2,3-dihydro-benzoimidazol-1-yl)-piperidin-1-yl]-ethyl}-amide. MS (APCI) M+531

Step 1 Preparation of 7-tert-Butoxycarbonylamino-heptanoic acid 2,5-dioxo-pyrrolidin-1-yl ester.

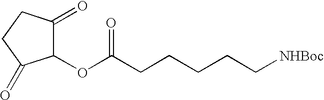

To a solution of 7-tert-butoxycarbonylamino-heptanoic acid (2.45 g 10 mmol) in methylene chloride (50 ml) was added N-hydroxysuccinamide (2.30 g, 20 mmol) followed by EDC (1,3-dimethylaminopropyl-3-ethylcarbodiimidehydrochloride, 3.83 g, 20 mmol). The resulting solution was stirred at room temperature overnight, and then diluted with methylene chloride (25 ml) and water (25 ml). The organic phase was washed first with saturated aqueous sodium bicarbonate solution (2 times, 25 ml each) and then saturated brine solution (2 times, 25 ml each), dried over magnesium sulfate, and concentrated to deliver 3.74 g (87%) product as a white solid.

Step 2 Preparation of 2-(7-tert-Butoxycarbonylamino-heptanoylamino)-3-(1H-indol-3-yl)-propionic acid.

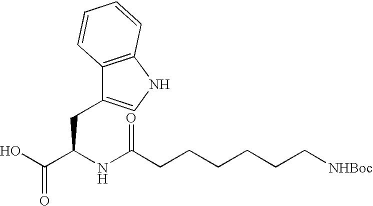

A solution of 7-tert-butoxycarbonylamino-heptanoic acid 2,5-dioxo-pyrrolidin-1-yl ester (3.73 g, 8.7 mmol), d-tryptophan (1.77 g, 8.7 mmol), and triethylamine (1.2 ml, 8.7 mmol) in 4:1 dioxane:water (25 ml) was stirred at 45° C. for 12 hours. The resulting solution was diluted with water (20 ml), and then acidified to pH 3 with citric acid solution. Product was extracted with ethyl acetate (2 washes×25 mL), washed with brine, dried over magnesium sulfate, and concentrated to afford 3.75 g (99%) product.

Step 3 Final synthesis of 7-Amino-heptanoic acid {1-1H-indol-3-ylmethyl)-2-oxo-2-[4-(2-oxo-2,3-dihydro-benzoimidazol-1-yl)-pi peridin-1-yl]-ethyl}-amide.

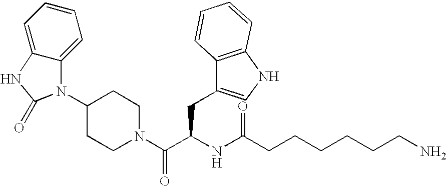

The following procedures are referred to as “general procedure A”: in subsequent examples. To a solution of 2-(7-tert-Butoxycarbonylamino-heptanoylamino)-3-(1H-indol-3-yl)-propionic acid (1.079 g, 2.5 mmol) and triethyl amine (700 μl, 5 mmol) in methylene chloride (20 ml) was added EDC (479 mg, 2.5 mmol) followed by 1-Piperidin-4-yl-1,3-dihydro-benzoimidazol-2-one (543 mg, 2.5 mmol). The mixture was stirred at room temperature overnight, diluted with methylene chloride (20 mL), and washed with water (3 times×25 mL) and brine (2 times×25 mL). The organic phase was dried over magnesium sulfate and concentrated to give 676 mg of a pale yellow solid that was subsequently stirred in a 4 M solution of HCl in dioxane (5 mL) for 20 minutes. The resulting mixture was evaporated to dryness and the product was triturated with ether to deliver 589 mg product as the HCl salt.

### Example 2

7-Amino-heptanoic acid {2-(1H-indol-3-yl)-1-[2-(1H-indol-3-yl)-ethylcarbamoyl]-ethyl}-amide. MS (APCI) M+474

This compound was prepared from 7-tert-butoxycarbonyl-amino-heptanoic acid 2,5-dioxo-pyrrolidin-1-yl ester (39 mg, 0.075 mmol) and tryptamine (17 mg, 0.075 mmol) according to general procedure A to give 20 mg product.

### Example 3

7-Amino-heptanoic acid [1-[2-(5-fluoro-1H-indol-3-yl)-ethylcarbamoyl]-2-(1H-indol-3-yl)-ethyl]-amide.

This compound was prepared from 7-tert-butoxycarbonyl-amino-heptanoic acid 2,5-dioxo-pyrrolidin-1-yl ester (39 mg, 0.075 mmol) and 5-flourotryptamine (16 mg, 0.075 mmol) according to general procedure A to give 18 mg product. MS (APCI) M+492

### Example 4

7-Amino-heptanoic acid (2-(1H-indol-3-yl)-1-{2-[2-(4-methoxy-phenyl)-1H-indol-3-yl]-ethylcarbamoyl}-ethyl)-amide.

This compound was prepared from was prepared from 7-tert-butoxycarbonyl-amino-heptanoic acid 2,5-dioxo-pyrrolidin-1-yl ester (39 mg, 0.075 mmol) and 2-(4-methoxyphenyl)-3-indoleethyl amine (17 mg, 0.075 mmol) according to general procedure A to give 16 mg product. MS (APCI) M+580

### Example 5

7-Amino-heptanoic acid [1-(indan-2-ylcarbamoyl)-2-(1H-indol-3-yl)-ethyl]-amide.

This compound was prepared from was prepared from 7-tert-butoxycarbonyl-amino-heptanoic acid 2,5-dioxo-pyrrolidin-1-yl ester (39 mg, 0.075 mmol) and aminoindane (12 mg, 0.075 mmol) according to general procedure A to give 15 mg product. MS (APCI) M+447.

### Example 6

7-Amino-heptanoic acid [1-[2-(6-fluoro-1H-indol-3-yl)-ethylcarbamoyl]-2-(1H-indol-3-yl)-ethyl]-amide.

This compound was prepared from 7-tert-butoxycarbonyl-amino-heptanoic acid 2,5-dioxo-pyrrolidin-1-yl ester (39 mg, 0.075 mmol) and 6-flourotryptamine (16 mg, 0.075 mmol) according to general procedure A to give 11 mg product. MS (APCI) M+492

### Example 7

7-Amino-heptanoic acid [2-[4-(2-chloro-dibenzo[b,f][1,4]oxazepin-11-yl)-pi perazin-1-yl]-l -(1H-indol-3-ylmethyl)-2-oxo-ethyl]-amide.

This compound was prepared from 7-tert-butoxycarbonyl-amino-heptanoic acid 2,5-dioxo-pyrrolidin-1-yl ester (43 mg, 0.1 mmol) and amoxapine (31, 0.1 mmol) according to general procedure A to give 60 mg product. MS (APCI) M+627

### Example 8

6-Amino-hexanoic acid {1-(1H-indol-3-ylmethyl)-2-oxo-2-[4-(2-oxo-2,3-dihydro-benzoimidazol-1-yl)-piperidin-1-yl]-ethyl}-amide.

This compound was prepared from 7-tert-butoxycarbonyl-amino-heptanoic acid 2,5-dioxo-pyrrolidin-1-yl ester (42 mg, 0.1 mmol) and 4-(2-keto-1-benzimidazolinyl)-piperidine (22 mg, 0.1 mmol) according to general procedure A to give 47 mg product. MS (APCI) M+517

### Example 9

7-Amino-heptanoic acid (2-(1H-indol-3-yl)-1-{([2-(1H-indol-3-yl)-ethyl]-methyl-carbamoyl}-ethyl)-amide.

This compound was prepared from 7-tert-butoxycarbonyl-amino-heptanoic acid 2,5-dioxo-pyrrolidin-1-yl ester (39 mg, 0.075 mmol) and N-methyl tryptamine (13 mg, 0.075 mmol) according to general procedure A to give 15 mg product. MS (APCI) M+488

### Example 10

7-Amino-heptanoic acid [1-[2-(5-fluoro-1H-indol-3-yl)-1-methyl-ethylcarbamoyl]-2-(1H-indol-3-yl)-ethyl]-amide MS (APCI) M+506.

### Example 11

7-Amino-heptanoic acid {2-(1H-indol-3-yl)-1-[2-(1H-indol-3-yl)-ethylcarbamoyl]-ethyl}-amide. MS (APCI) M+474

### Example 12

7-Amino-heptanoic acid [1-[2-(6-benzyloxy-1H-indol-2-yl)-ethylcarbamoyl]-2-(1H-indol-3-yl)-ethyl]-amide MS (APCI) M+580

### Example 13

6-Amino-hexanoic acid [1-(4-ethyl-benzylcarbamoyl)-2-(1H-indol-3-yl)-ethyl]-amide. MS (APCI) M+435

### Example 14

1-{1-[2-(7-Amino-heptanoyl)-2,3,4,9-tetrahydro-1H-arboline-3-carbonyl]-piperidin-4-yl}-1,3-dihydro-benzoimidazol-2-one. MS (APCI) M+543

### Example 15

7-Amino-heptanoic acid (2-(1H-indol-3-yl)-1-{[2-(1H-indol-3-yl)-ethyl]-methyl-carbamoyl}-ethyl)-amide. MS (APCI) M+488

### Example 16

7-Amino-heptanoic acid {2-(1H-indol-3-yl)-1-[2-(5-methoxy-1H-indol-3-yl)-ethylcarbamoyl]-ethyl}-amide. MS (APCI) M+504

### Example 17

7-Amino-heptanoic acid [1-[4-(biphenyl4-carbonyl)-piperazine-1-carbonyl]-2-(1H-indol-3-yl)-ethyl]-amide. MS (APCI) M+580

### Example 18

7-Amino-heptanoic acid [2-(1H-indol-3-yl)-1-(4-phenyl-butylcarbamoyl)-ethyl]-amide. MS (APCI) M+463

### Example 19

7-Amino-heptanoic acid [1-[2-(2-fluoro-phenyl)-ethylcarbamoyl]-2-(1H-indol-3-yl)-ethyl]-amide. MS (APCI) M+453

### Example 20

CP-634829-01: 7-Amino-heptanoic acid (1-(1H-indol-3-ylmethyl)-2-oxo-2-[4-(2-oxo-2,3-dihydro-benzoimidazol-1-yl)-piperidin-1-yl]-ethyl}-amide. MS (APCI) M+531

### Example 21

7-Amino-heptanoic acid [2-(1H-indol-3-yl)-1-(1H-indol-5-ylcarbamoyl)-ethyl]-amide. MS (APCI) M+446

### Example 22

7-Amino-heptanoic acid [2-(1H-indol-3-yl)-1-(3-phenyl-propylcarbamoyl)-ethyl]-amide. MS (APCI) M+449

### Example 23

2-(7-Amino-heptanoylamino)-3-(1H-indol-3-yl)-propionic acid biphenyl-4-ylmethyl ester. MS (APCI) M+498

### Example 24

7-Amino-heptanoic acid [2-(1H-indol-3-yl)-1-(2-phenyl-cyclopropylcarbamoyl)-ethyl]-amide. MS (APCI) M+447

### Example 25

6-Amino-hexanoic acid [1-(3-ethyl-benzylcarbamoyl)-2-(1H-indol-3-yl)-ethyl]-amide. MS (APCI) M+435

### Example 26

7-Amino-heptanoic acid {2-(1H-indol-3-yl)-1-[4-(toluene-4-sulfonyl)-piperazine-1-carbonyl]-ethyl}-amide. MS (APCI) M+554

### Example 27

7-Amino-heptanoic acid [1-(4-[4,4-bis-(4-fluoro-phenyl)-butyl]-piperazine-1-carbonyl}-2-(1H-indol-3-yl)-ethyl]-amide. MS (APCI) M+644

### Example 28

8-Amino-octanoic acid {1-(1H-indol-3-ylmethyl)-2-oxo-2-[4-(2-oxo-2,3-dihydro-benzoimidazol-1-yl)-piperidin-1 -yl]-ethyl}-amide. MS (APCI) M+545

### Example 29

6-Amino-hexanoic acid {1-(1H-indol-3-ylmethyl)-2-oxo-2-[4-(2-oxo-2,3-dihydro-benzoimidazol-1-yl)-piperidin-1-yl]-ethyl}-amide. MS (APCI) M+517

### Example 30

7-Amino-heptanoic acid [2-(1H-indol-3-yl)-1-(2-p-tolyl-ethylcarbamoyl)-ethyl]-amide. MS (APCI) M+449

Biological Assays

Various types of somatostain agonists are well known in the art, and the capacity of a compound of the present invention to act as an agonist, an antagonist, or as either, depending on physiological circumstances, can be predicted from the assays which are known in the art and/or described below. For example, measurement of cyclic-AMP, growth hormone release, microphysiometry responses, cell proliferation or protein kinase activity can be measured in cultured pituitary cells, cell lines or other cells such as neuroblastoma cells that express somatostatin receptors, and cells transfected with recombinant somatostatin receptors including transfected yeast cells. (Y. C. Patel et al., Biochemical & Biophysical Research Communications, 198(2), pp. 605-612, 1994; M. G. Cattaneo et al., FEBS Letters, 397(2-3), pp. 164-168, 1996; J. A. Koenig et al., British Joumal of Pharmacology, 120(1), pp. 45-51, 1997; D. Djordjijevic et al., Endocrinology, 139(5), pp. 2272-2277, 1998; W. R. Baumbach et al., Molecular Pharmacology, 54(5), pp. 864-73, 1998).

Generally, somatostatin or agonists thereof demonstrate inhibitory activity, hence a stimulus is first applied (e.g. forskolin for cyclic-AMP) and the inhibitory effect of somatostatin observed. Antagonists reverse the inhibitory effects of somatostatin.

The ability of compounds of formula (I), and the pharmaceutically acceptable salt, solvates or hydrate thereof (hereinafter referred to as the compounds of the present invention) to act as somatostatin antagonists, or agonists, and consequently to demonstrate their effectiveness in the treatment of disease states, is shown by the following assays.

### Example 31

bovine (“b”)sst2 binding assay

The present example describes an assay for binding of pharmaceutically useful somatostatin agonists and antagonists at the bovine sst2 receptor.

The methods for culturing Neuro2A cells and measuring competitive binding potency (IC₅₀) were similar to those described by J. A. Koenig et al., “Somatostatin receptors in Neuro2A neuroblastoma cells: operational characteristics”, British J. Pharmacol., 120, 45-51, 1997, with the following modifications.

Binding assays were conducted 72 hours after transiently transfecting the Neuro2A cells with a plasmid (PCI-bsst2) containing an insert coding for the bovine sst2 receptor, placed downstream of the cytomegalovirus promoter. In the transfection step, 6.5×10⁶ Neuro2A cells were added in 35 ml of media to each tissue culture flask (162 cm² surface area). The next day, transfection was conducted using Fugene 6 (Boehringer Mannheim, 1 814 443) according to the manufacturer's directions. The Fugene 6 (30 μl/flask) was equilibrated with 8 μg of PCI-bsst2 plasmid, and added to the Neuro2A cells in the absence of fetal bovine serum. After 3 hours, fresh serum-containing media was added. The assay buffer was modified to contain 50 mM HEPES, 5 mM MgCl₂, 1 mg/ml bovine serum albumin (BSA), 0.02 mg/ml bacitracin, and 10 gM each of aprotinin, leupeptin and AEBSF. The transfected Neuro2A cells were dissociated in the absence of trypsin/EDTA, in ice cold assay buffer (5.5 ml/flask), and cells were homogenized in a 55 ml Wheaton Dounce homogenizer (15-20 strokes). Membrane preparations were stored in aliquots at −70° C. Competitive binding assays and separation of bound from free radioactivity were conducted in polyethyleneimine-soaked Millipore 96 Well GF/C Filterplates, (MAFC NOB10). An amount of membrane was used that bound approximately 20% of [¹²⁵I]-somatostatin 14 tracer (Amersham, IM161), which was added to all wells at 15,000 cpm/well (approximately 15 nCi/well). Somatostatin was included in each experiment as positive control, at 7 concentrations from 0.0042 to 1.667 nM, and test compounds were included at 7 concentrations from 33 nM to 13.33 μM. The reaction volume was 300 μl and the incubation was conducted for 1 hour at 37° C. Non-specific binding was defined using 0.83 μM somatostatin 14. The incubation was terminated by vacuum filtration through the glass fiber plate bottom, followed with a 250 μl wash with assay buffer minus BSA and protease inhibitors. The plate bottom was then sealed, scintillation fluid was added (Wallac Supermix, 250 μl/well), and radioactivity was measured in a 96 well microtiter liquid scintillation counter.

IC₅₀ values are determined by polynomial regression and analzyed using a MACRO program. An IC₅₀ value of less than about 5 μM is preferred.

### Example 32

Rat Pituitary Assay for Somatostatin Receptor Antagonists

This assay is designed to quantitate the activity of antagonists of somatostatin that interact directly at the somatostatin receptor. The assay facilitates discovery of agents which increase growth hormone secretion by modulating the inhibitory effects of somatostatin. As aforementioned, somatostatin (also abbreviated SRIF) inhibits GH secretion in the anterior pituitary by binding to a high affinity membrane-bound (and G-protein coupled) receptor which is coupled negatively to adenyl cyclase, thereby reducing intracellular levels of cAMP that would otherwise facilitate, for example, secretion/release of GH from cytoplasmic granules. Vasoactive intestinal peptide (VIP) is one of several endogenous peptides that stimulates GH secretion by binding to a high affinity membrane-bound receptor coupled to a G protein-dependent signal transduction pathway. VIP activates adenylate cyclase and produces increased intracellular cAMP levels. These peptides may be involved in the coordinate regulation of GH secretion under physiologic conditions and be mediated through cAMP. The cell line used in the screen is a clonal pituitary cell that synthesizes and secretes GH in response to VIP and SRIF, and many other regulatory hormones, as expected for normal pituitary cells. The screen is designed to quantitate the ability of test agents to reverse SRIF's inhibition of the elevated intracellular cAMP levels produced by VIP.

In particular, cyclic AMP (cAMP) content of the pituitary cell line GH₄C₁ was used to differentiate somatostatin agonists from antagonists. The method was similar to that described by L. J. Dorflinger et al. (“Somatostatin inhibits vasoactive intestinal peptide-stimulated cyclic adenosine monophosphate accumulation in GH pituitary cells”, Endocrinology ,113, pp. 1541-50, 1983 ) with the following modifications. Aliquots (50 μl) of GH₄C₁ cell suspension at 1-2 million cells/ml were added to 50 μl of each solution of test compound in Adenylyl Cyclase Activation FlashPlate® Assay plates from NEN™ Life Science Products (catalog SMP004A). Putative somatostatin agonists or antagonists were typically tested at concentrations of 10, 1 and 0.1 μM, in the presence of 100 nM vasoactive intestinal peptide (VIP; Sigma V3628) and 10 nM somatostatin 14 (cell culture tested, Sigma S1763). The FlashPlates®, which are coated with antibody against cAMP and contain scintillant integral to the plastic, are supplied as part of a kit with all necessary reagents to estimate cAMP content of whole cell preparations, including Stimulation Buffer, Detection Buffer, cAMP Standard, and [¹²⁵I]-cAMP Tracer. This afforded a convenient way to conduct a homogenous immunoradiometric assay of cAMP content in cells lysed in situ, following incubation of the cells with test compound. cAMP content in the GH₄C₁ cells was determined according to the manufacturer's instructions, by comparison with standards at concentrations from 10 to 1,000 nM cAMP. In this assay, VIP increased cAMP content of the GH₄C₁ cells, and somatostatin caused a partial inhibition. Test compounds acting as somatostatin antagonists were detected by their tendancy to increase cAMP content in comparison to control wells containing VIP and somatostatin but no test compound. Somatostatin agonists conversely decreased cAMP content.

IC₅₀ values are determined by polynomial regression and analzyed using a MACRO program. An IC₅₀ value of less than about 5 μM is preferred.

### Example 33

Effect of a somatostatin antagonist on GH release in 12 kg pigs

Studies indicate that concentrations of GH increase in small pigs within 10 minutes of administration of somatostatin antagonists, and then return to pre-treatment levels within 40 minutes post-administration.

The following protocol describes the effects of various doses of a somatostatin antagonist on release of endogenous porcine GH (or pST, porcine somatatrophin). Methods used to evaluate effects of compounds on plasma GH concentrations in barrows (castrated male pigs) were similar to those reported by M. J. Estienne et al., “Methyl-D,L-aspartate-induced growth hormone secretion in barrows: possible mechanisms of action”, Journal of Animal Science ,74, pp. 597-602, 1996, with the following modifications. Forty cross-bred barrows weighing approximately 12 kg were acclimatized for 2 days at 10 pigs per 36 sq. ft. pen, 4 pens per study, with feed (PS-9 swine starter diet) and water provided ad libitum. To enhance uniformity, two pigs/pen were eliminated based on being smallest or largest, or for health reasons, bringing the group size to 8 pigs/treatment. An equal number of pigs in each pen received 1 of 4 possible treatments at random, i.e. one of 3 doses of test compound or diluent alone. Compounds diluted in approximately 1 ml/pig sterile saline were administered by intramuscular injection into the rear leg (ham), about 1 minute after collection of the first blood sample into 7 ml heparinized evacuated tubes via jugular venepuncture. Blood samples were similarly collected at 10 minute intervals up to 40 minutes after injection of test compound or diluent. Plasma was separated by centrifugation and frozen at −20° C.).

### Example 34

RIA Procedure for determination of GH levels in plasma.

The present assay is used to determine GH levels (for example, porcine GH or canine GH) in plasma samples.

The double antibody radioimmunoassay (RIA) used to determine porcine GH concentrations in plasma samples was similar to that described by Y. N. Sinha et al., “Studies of GH secretion in mice by a homologous radioimmunoassay for mouse GH”, Endocrinology ,91, pp.784-92, 1972, and that of F. Cocola et al., “A rapid radioimmunoassay method of growth hormone in dog plasma”, Proceedings of the Society for Experimental Biology and Medicine ,151, pp. 140-14, 1976. Modifications were as follows. Native porcine GH (pGH) for radioiodination as tracer, canine GH for use as standard (cGH; AFP-1983B; the aminoacid sequence of canine and porcine GH are the same), and primary antibody (monkey anti-cGH; AFP-21452) were supplied by A. F. Parlow, Harbor UCLA Medical Center. Recombinant porcine GH from Biogenesis was alternatively used for radioiodination as tracer. Radioiodinations were conducted by Biomedical Technologies Inc, Stoughton, Mass. Primary antibody (1:50,000 or 1:100,000 final dilution), normal monkey serum (ICN 55988; 1:1,000 final dilution), and plasma sample or standard (0.08 to 2.5 ng cGH/tube) were mixed and incubated for 2 hours at ambient temperature, then tracer (10,000 cpm/tube) was added and the incubation continued for a further 20 hours at ambient temperature in a total volume of 500 μl. Secondary antibody (goat anti-monkey lgG ICN 55418; final dilution 1:160) and polyethyleneglycol 8,000 (final concentration 44 mg/ml) were added and mixed in a final volume of 1.6 ml. Tubes were incubated at 4° C. for 2 hours with shaking, then they were centrifuged, supernates discarded, and the gamma-emission of the pellets determined.

Plasma growth hormone concentrations, expressed as ng/ml, were calculated from the standard line following log-logit transformation.

1. A compound according to the formula A-B-Z-W (formula 1), or a pharmaceutically acceptable salt, solvate, or hydrate thereof; wherein
A is selected from
(a) (C₆-C₁₀)aryl-, selected from phenyl or naphthyl; or
(b) (C₁-C₉)heteroaryl-, selected from the group consisting of furyl-, thienyl-thiazolyl-, pyrazolyl-, isothiazolyl-, oxazolyl-, isoxazolyl-, pyrrolyl-, triazolyl-, tetrazolyl-, imidazolyl-, 1,3,5-oxadiazolyl-, 1,2,4-oxadiazolyl-, 1,2,3-oxadiazolyl-, 1,3,5-thiadiazolyl-, 1,2,3-thiadiazolyl-, 1,2,4-thiadiazolyl-, pyridyl-, pyrimidyl-, pyrazinyl-, pyridazinyl-, 1,2,4-triazinyl-, 1,2,3-triazinyl-, 1,3,5-triazinyl-, pyrazolo[3,4-b]pyridinyl-, cinnolinyl-, pteridinyl-, purinyl-, 6,7-dihydro-5H-[1]pyrindinyl-, benzo[b]thiophenyl-, 5, 6, 7, 8-tetrahydro-quinolin-3-yl, benzoxazolyl-, benzothiazolyl-, benzisothiazolyl-, benzisoxazolyl-, benzimidazolyl-, thianaphthenyl-, isothianaphthenyl-, benzofuranyl-, isobenzofuranyl-, isoindolyl-, indolyl-, indolizinyl-, indazolyl-, isoquinolyl- quinolyl-, phthalazinyl-, quinoxalinyl-, quinazolinyl-, and benzoxazinyl-;
wherein said A group (a) or (b) is optionally substituted by zero to seven, preferably zero to five groups, each independently selected from:
hydroxy, halo, amino, trifluoromethyl-, carboxy, (C₁-C₆)alkoxy-, (C₁-C₆)acyloxy-, (C₁-C6)alkylamino-, ((C₁-C6)alkyl)₂amino-, (C₁-C₆)acylamino-, cyano, nitro, (C₁-C₆)alkyl-, (C₂-C₆)alkenyl-, (C₂-C₆)alkynyl-, (C₁-C₆)acylamino-, cyano(C₁-C₆)alkyl-, trifluoromethyl(C₁-C₆)alkyl-, nitro(C₁-C6)alkyl-, (C₁-C₃)alkyl(difluoromethylene)(C₁-C₃)alkyl-, (C₁-C₆)acylamino(C₁-C₆)alkyl-, (C₁-C₆)alkoxy(C₁-C₆)acylamino-, amino(C₁-C₆)acyl-, amino(C₁-C₆)acyl(C₁-C₆)alkyl-, (C₁-C₆)alkylamino(C₁-C₆)acyl-, ((C₁-C₆)alkyl)₂amino(C₁-C₆)acyl-, (C₃-C₁₀)cycloalkyl(C₁-C₆)alkyl-, (C₁-C₆)acyloxy(C₁-C₆)alkyl-, (C₂-C₆)alkoxy(C₁-C₆)alkyl-, piperazinyl(C₁-C₆)alkyl-, (C₁-C₆)acylamino(C₁-C₆)alkyl-, (C₆-C₁₀)aryl(C₁-C₆)alkoxy(C₁-C₆)alkyl-, (C₁-C₉)heteroaryl(C₁-C₆)alkoxy(C₁-C₆)alkyl-, (C₁-C₆)alkylthio(C₁-C₆)alkyl-, (C₆-C₁₀)arylthio(C₁-C6)alkyl-, (C₁-C₆)alkylsulfinyl(C₁-C₆)alkyl-, (C₆-C₁₀)arylsulfinyl(C₁-C₆)alkyl-, (C₁-C6)alkylsulfonyl(C₁-C₆)alkyl-, (C₆-C₁₀)arylsulfony((C₁-C₆)alkyl-, amino(C₁-C₆)alkyl-, (C₁-C6)alkylamino(C₁-C₆)alkyl-, (C₁-C₆)alkyl(difluoromethylene)-, (C₁-C₃)alkyl(difluoromethylene)(C₁-C₃)alkyl-, (C₁-C₆)alkoxy(C₁-C₆)acyl-, (C₁-C₆)alkylamino(C₁-C₆)acyl-, ((C₁-C₆)alkyl)₂amino(C₁-C₆)acyl-, (C₆-C₁₀)aryl-, (C₁-C₉)heteroaryl-, (C₆-C₁₀)aryl(C₁-C₆)alkyl-, (C₁-C₉)heteroaryl(C₁-C₆)alkyl-, (C₆-C₁₀)aryl(C₆-C₁₀)aryl-, (C₆-C₁₀)aryl(C₆-C₁₀)aryl(C₁-C₆)alkyl- (C₃-C₁₀)cycloalkyl-, (C3-C₆)cycloalkyl(C₁-C₆)alkyl-, (C₃-C₁₀)heterocycloalkyl-, (C₃-C₁₀)heterocycloalkyl(C₁-C₆)alkyl-, hydroxy(C₂-C₆)alkyl-, (C₁-C₆)acyloxy(C₂-C₆)alkyl-, (C₁-C₆)alkoxy(C₂-C₆)alkyl-, piperazinyl(C₁-C₆)alkyl-, (C₁-C₆)acylamino(C₁-C₆)alkyl-, (C₆-C₁₀)aryl(C₁-C₆)alkoxy(C₁-C₆)alkyl-, (C₁-C₉)heteroaryl(C₁-C₆)alkoxy(C₁-C₆)alkyl-, (C₁-C₆)alkylthio(C₁-C₆)alkyl-, (C6-C₁₀)arylthio(C₁-C₆)alkyl-, (C₁-₆)alkylsulfinyl(C₁-C₆)alkyl-, (C₆-C₁₀)arylsulfinyl(C₁-C₆)alkyl-, (C₁-C₆)alkylsulfonyl(C₁-C₆)alkyl-, (C₆-C₁₀)arylsulfonyl(C₁-C6)alkyl-, amino(C₁-C₆)alkyl-, (C₁-C₆)alkylamino(C₁-C₆)alkyl-, and ((C₁-C₆)alkyl)₂amino(C₁-C₆)alkyl-;
B is selected from
(a) O, NH, NR¹⁰, —(CH2)ₖ—O—, —(CH₂)ₖ—N—, and —(CH₂)_{k—NR}¹⁰—, where R¹⁰ is (C₁-C₆)alkyl, and where k is 1 to 6 in each case, or 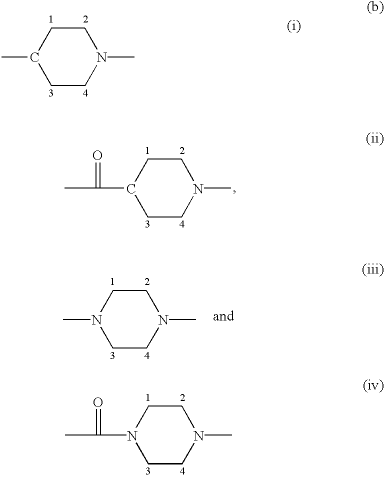
where said group (i) through (iv) is optionally substituted by 1 to 4, preferably 1 to 2, groups selected from (C₁-C₆)alkyl, halo, and (C₁-C₆)alkyl optionally substituted by 1 to 3 halo atoms, wherein one of carbon atoms C₁, C₂, C₃ and C₄ of said piperidine or piperazine group is optionally replaced by a carbonyl group;
Z is selected from groups (i) to (vii): 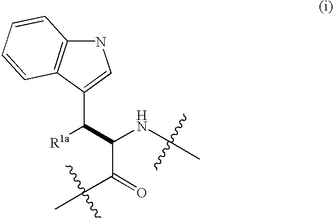
where R^{1a} is H, or (C₁-C₆)alkyl optionally substituted by 1 to 3 halo atoms; 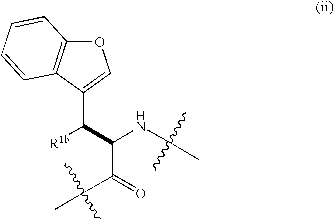
where R^{1b} is H, or (C₁-C₆)alkyl optionally substituted by 1 to 3 halo atoms; 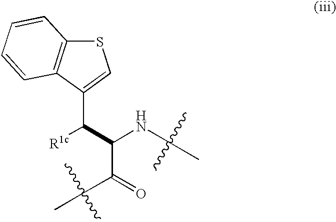
where R^{1c} is H, or (C₁-C₆)alkyl optionally substituted by 1 to 3 halo atoms; 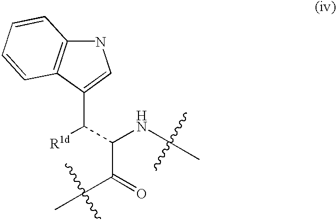
wherein R^{1d} is H, or (C₁-C₆)alkyl optionally substituted by 1 to 3 halo atoms; 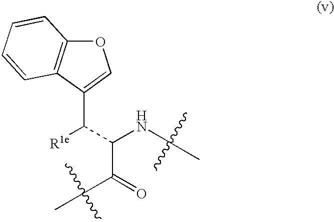
where R^{1e} is H, or (C₁-C₆)alkyl optionally substituted by 1 to 3 halo atoms; 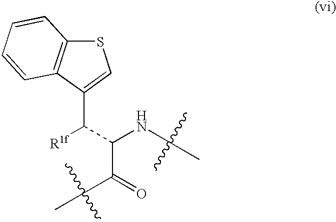
where R^{1f} is H, or (C₁-C₆)alkyl optionally substituted by 1 to 3 halo atoms; 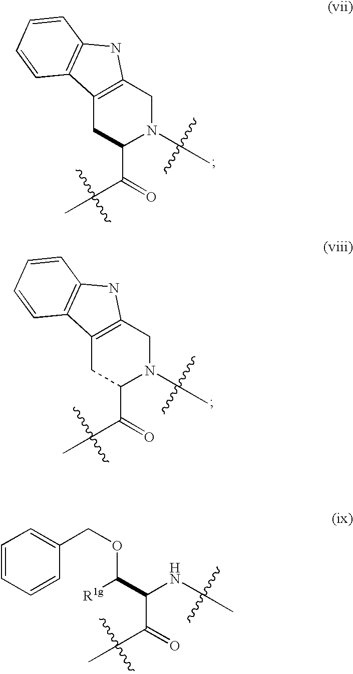
where R^{1g} is H, or (C₁-C₆)alkyl optionally substituted by 1 to 3 halo atoms; 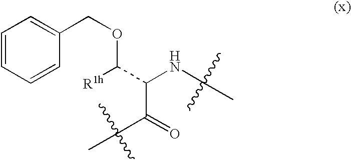
where R^{1h} is H, or (C₁-C₆)alkyl optionally substituted by 1 to 3 halo atoms; 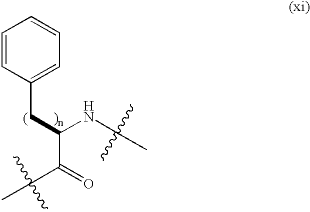
where n is 0 to 6, preferably 1 to 3; 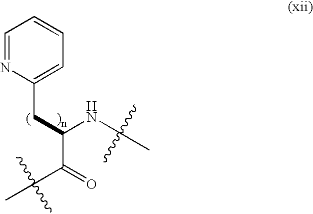
where n is 0 to 6, preferably 1 to 3; 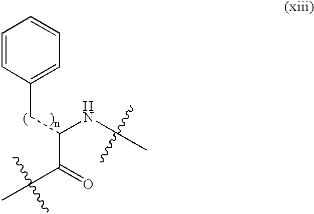
where n is 0 to 6, preferably 1 to 3; and 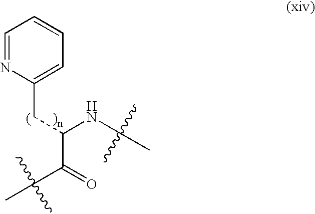
where n is 0 to 6, preferably 1 to 3; and
W is selected from: 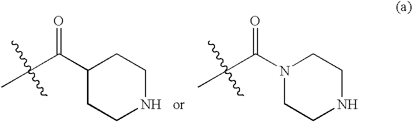
wherein one or more ring carbons of said piperidine or piperazine ring is optionally substituted by (C₁-C₆)alkyl or halo; 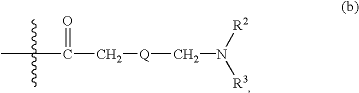
wherein Q is selected from the group consisting of:
(i) (C₆-C₁₀)aryl-, selected from phenyl or naphthyl;
(ii) (C₁-C₉)heteroaryl-, selected from the group consisting of furyl-, thienyl-thiazolyl-, pyrazolyl-, isothiazolyl-, oxazolyl-, isoxazolyl-, pyrrolyl-, triazolyl-, tetrazolyl-, imidazolyl-, 1,3,5-oxadiazolyl-, 1,2,4-oxadiazolyl-, 1,2,3-oxadiazolyl-, 1,3,5-thiadiazolyl-, 1,2,3-thiadiazolyl-, 1,2,4-thiadiazolyl-, pyridyl-, pyrimidyl-, pyrazinyl-, pyridazinyl-, 1,2,4-triazinyl-, 1,2,3-triazinyl-, 1,3,5-triazinyl-, pyrazolo[3,4-b]pyridinyl-, cinnolinyl-, pteridinyl-, purinyl-, 6,7-dihydro-5H-[1]pyrindinyl-, benzo[b]thiophenyl-, 5, 6, 7, 8-tetrahydro-quinolin-3-yl, benzoxazolyl-, benzothiazolyl-, benzisothiazolyl-, benzisoxazolyl-, benzimidazolyl-, thianaphthenyl-, isothianaphthenyl-, benzofuranyl-, isobenzofuranyl-, isoindolyl-, indolyl-, indolizinyl-, indazolyl-, isoquinolyl- quinolyl-, phthalazinyl-, quinoxalinyl-, quinazolinyl-, and benzoxazinyl-;
(iii) (C₃-C₁₀)cycloalkyl that is selected from the group consisting of cyclopropyl-, cyclobutyl-, cyclopentyl-; cyclohexyl-, cycloheptyl-, cyclopropenyl-, cyclobutenyl-cyclopentenyl-, cyclohexenyl-, cycloheptenyl-, 1,3-cyclobutadienyl-, 1,3-cyclopentadienyl-, 1,3-cyclohexadienyl-, 1,4-cyclohexadienyl- 1,3-cycloheptadienyl-, 1,4-cycloheptadienyl-, 1,3,5-cycloheptatrienyl- bicyclo[3.2.1]octane, bicyclo [2.2.1] heptane and the norborn-2-ene unsaturated form thereof; and
(iv) (C₃-C₁₀) heterocycloalkyl that is selected from the group consisting of pyrrolidinyl-, tetrahydrofuranyl- dihydrofuranyl-, tetrahydropyranyl-, pyranyl-, thiopyranyl-, aziridinyl-, oxiranyl-, methylenedioxyl-, chromenyl-, isoxazolidinyl-, 1,3-oxazolidin-3-yl-isothiazolidinyl-, 1,3-thiazolidin-3-yl-, 1,2-pyrazolidin-2-yl-, 1,3-pyrazolidin-1-yl-, piperidinyl-, thiomorpholinyl-, 1,2-tetrahydrothiazin-2-yl-, 1,3-tetrahydrothiazin-3-yl-, tetrahydrothiadiazinyl-, morpholinyl-, 1,2-tetrahydrodiazin-2-yl-, 1,3-tetrahydrodiazin-1-yl-, tetrahydroazepinyl-, piperazinyl-, and chromanyl;
wherein R² and R³ thereof are each independently selected from H, (C₁-C₈)alkyl, and phenyl(CH₂)—, wherein said alkyl and phenyl groups are optionally substituted by one or more halo; and 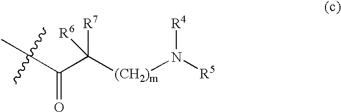
wherein m is 1 to 7, R⁴ and R⁵ are each independently selected from the group consisting of 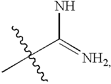
H, (C₁-C₆)alkyl, and phenyl(CH₂)—, wherein said alkyl and phenyl groups are optionally substituted by one or more halo atoms, and
R⁶ and R⁷ are each independently selected from
(i) H, CH₃—, NH₂—, and CH₃C(O)—NH—, or from 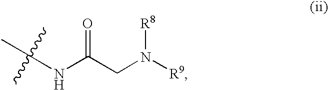
wherein R⁸ and R⁹ are each independently selected from H, (C₁-C₈)alkyl, and phenyl(CH₂)—, and said alkyl and phenyl groups are optionally substituted by one or more halo atoms.

2. The compound of claim 1, wherein group A is a (C₆-C₁₀)aryl- group selected from phenyl and naphthyl.

3. The compound of claim 1, wherein group A is a (C_{1-C}₉)heteroaryl- group that is selected from the group consisting of furyl-, thienyl-, thiazolyl-, pyrazolyl-, isothiazolyl-, oxazolyl-, isoxazolyl-, pyrrolyl-, triazolyl-, tetrazolyl-, imidazolyl-, 1,3,5-oxadiazolyl-, 1,2,4-oxadiazolyl-, 1,2,3-oxadiazolyl-, 1,3,5-thiadiazolyl-, 1,2,3-thiadiazolyl-, 1,2,4-thiadiazolyl-, pyridyl-, pyrimidyl-, pyrazinyl-, pyridazinyl-, 1,2,4-triazinyl-, 1,2,3-triazinyl-, 1,3,5-triazinyl-, pyrazolo[3,4-b] pyridinyl-, cinnolinyl-, pteridinyl-, purinyl-, 6,7-dihydro-5H-[1] pyrindinyl-, benzo[b] thiophenyl-, 5, 6, 7, 8-tetrahydro-quinolin-3-yl-, benzoxazolyl-, benzothiazolyl-, benzisothiazolyl-, benzisoxazolyl-, benzimidazolyl-, thianaphthenyl-, isothianaphthenyl-, benzofuranyl-, isobenzofuranyl-, isoindolyl-, indolyl-, indolizinyl-, indazolyl-, isoquinolyl-, quinolyl-, phthalazinyl-, quinoxalinyl-, quinazolinyl-, and benzoxazinyl-.

4. The compound of claim 1, wherein group A is optionally substituted by one to five groups, each independently selected from the group consisting of hydroxy, halo, amino, trifluoromethyl, carboxy, (C₁-C₆)alkoxy-, (C₁-C₆)acyloxy-, (C₁-C₆)alkylamino-, ((C₁-C₆)alkyl)₂amino-, (C₁-C₆)acylamino-, cyano, nitro, (C₁-C₆)alkyl-, (C₂-C₆)alkenyl-, (C₂-C₆)alkynyl-, (C₁-C₆)acylamino-, cyano(C₁-C₆)alkyl-, trifluoromethyl(C₁-C₆)alkyl-, nitro(C₁-C₆)alkyl-, (C₁-C₃)alkyl(difluoromethylene)(C₁-C₃)alkyl-, (C₁-C6)acylamino(C₁-C₆)alkyl-, (C₁-C₆)alkoxy(C₁-C₆)acylamino-, amino(C₁-C₆)acyl-, amino(C₁-C₆)acyl(C₁-C₆)alkyl-, (C₁-C₆)alkylamino(C₁-C₆)acyl-, ((C₁-C₆)alkyl)₂amino(C₁-C₆)acyl-, (C₃-C₁o)cycloalkyl(C₁-C₆)alkyl-, (C₁-C₆)acyloxy(C₁-C₆)alkyl-, (C₂-C₆)alkoxy(C₁-C₆)alkyl-, piperazinyl(C₁-C₆)alkyl-, (C₁-C₆)acylamino(C₁-C₆)alkyl-, (C₆-C₁₀)aryl(C₁-C₆)alkoxy(C₁-C₆)alkyl-, (C₂-C₉)heteroaryl(C₁-C6)alkoxy(C₁-C₆)alkyl-, (C₁-C₆)alkylthio(C₁-C₆)alkyl-, (C₆-C₁₀)arylthio(C₁-C₆)alkyl-, (C₁-C₆)alkylsulfinyl(C₁-C₆)alkyl- (C₆-C₁₀)arylsulfinyl(C₁-C₆)alkyl-, (C₁-C₆)alkylsulfonyl(C₁-C₆)alkyl-, (C₆-C₁₀)arylsulfonyl(C₁-C₆)alkyl-, amino(C₁-C₆)alkyl-, (C₁-C6)alkylamino(C₁-C₆)alkyl-, (C₁-C₆)alkyl(difluoromethylene)-, (C₁-C₃)alkyl(difluoromethylene)(C₁-C₃)alkyl-, (C₁-C₆)alkoxy(C₁-C₆)acyl-, (C₁-C₆)alkylamino(C₁-C₆)acyl-, ((C₁-C₆)alkyl)₂amino(C₁-C₆)acyl-, (C₆-C₁₀)aryl-, (C₅-C₉)heteroaryl-, (C₆-C₁₀)aryl(C₁-C₆)alkyl-, (C₂-Cg)heteroaryl(C₁-C₆)alkyl-, (C₆-C₁₀)aryl(C₆-C₁₀)aryl-, (C₆-C₁₀)aryl(C₆-C₁₀)aryl(C₁-C₆)alkyl- (C₃-C₁₀)cycloalkyl-, (C₃-C₆)cycloalkyl(C₁-C₆)alkyl-, (C₃-C₁₀)heterocycloalkyl-, (C₃-C₁₀)heterocycloalkyl(C₁-C₆)alkyl-, hydroxy(C₂-C₆)alkyl-, (C₁-C₆)acyloxy(C₂-C₆)alkyl-, (C₁-C₆)alkoxy(C₂-C₆)alkyl-, piperazinyl(C₁-C₆)alkyl-, (C₁-C₆)acylamino(C₁-C₆)alkyl-, (C₆-C₁₀)aryl(C₁-C₆)alkoxy(C₁-C₆)alkyl-, (C₂-C₉)heteroaryl(C₁-C₆)alkoxy(C₁-C₆)alkyl-, (C₁-C₆)alkylthio(C₁-C₆)alkyl-, (C₆-C₁₀)arylthio(C₁-C₆)alkyl-, (C₁-C₆)alkylsulfinyl(C₁-C₆)alkyl-, (C₆-C₁₀)arylsulfinyl(C₁-C₆)alkyl-, (C₁-C₆)alkylsulfonyl(C₁-C₆)alkyl-, (C₆-C₁₀)arylsulfonyl(C₁-C₆)alkyl-, amino(C₁-C₆)alkyl-, (C₁-C6)alkylamino(C₁-C₆)alkyl-, and ((C₁-C₆)alkyl)₂amino(C₁-C₆)alkyl.

5. The compound of claim 1, wherein group Q of group W, option (b), is a (C₆-C₁₀)aryl- group selected from phenyl and naphthyl.

6. The compound of claim 1, wherein group Q of group W, option (b), is a (C₁-C₉)heteroaryl- group that is selected from the group consisting of furyl-, thienyl- thiazolyl-, pyrazolyl-, isothiazolyl-, oxazolyl-, isoxazolyl-, pyrrolyl-, triazolyl-, tetrazolyl-, imidazolyl-, 1,3,5-oxadiazolyl- 1,2,4-oxadiazolyl-, 1,2,3-oxadiazolyl-, 1,3,5-thiadiazolyl-, 1,2,3-thiad iazolyl-, 1,2,4-thiadiazolyl-, pyridyl-, pyrimidyl-, pyrazinyl-, pyridazinyl-, 1,2,4-triazinyl-, 1,2,3-triazinyl-, 1,3,5-triazinyl-, pyrazolo[3,4-b] pyridinyl-, cinnolinyl-, pteridinyl-, purinyl-, 6,7-dihydro-5H-[1] pyrindinyl-, benzo[b] thiophenyl-, 5, 6, 7, 8-tetrahydro-quinolin-3-yl-, benzoxazolyl-, benzothiazolyl-, benzisothiazolyl-, benzisoxazolyl-, benzimidazolyl-, thianaphthenyl-, isothianaphthenyl-, benzofuranyl-, isobenzofuranyl-, isoindolyl-, indolyl-, indolizinyl-, indazolyl-isoquinolyl-, quinolyl-, phthalazinyl-, quinoxalinyl-, quinazolinyl-, and benzoxazinyl-.

7. The compound of claim 1, wherein group Q of group W, option (b), is a (C₃-C₁₀)cycloalkyl- group that is selected from the group consisting of cyclopropyl- cyclobutyl-, cyclopentyl-, cyclohexyl-, cycloheptyl- cyclopropenyl-, cyclobutenyl-, cyclopentenyl-, cyclohexenyl-, cycloheptenyl-, 1,3-cyclobutadienyl-, 1,3-cyclopentadienyl-, 1,3-cyclohexadienyl-, 1,4-cyclohexadienyl-, 1,3-cycloheptadienyl-, 1,4-cycloheptadienyl-, 1,3,5-cycloheptatrienyl-, bicyclo[3.2.1] octane-, bicyclo [2.2.1] heptane-, and the norborn-2-ene unsaturated form thereof.

8. The compound of claim 1, wherein group Q of group W, option (b), is a (C₃-C₁₀)heterocycloalkyl- group that is selected from the group consisting of pyrrolidinyl-, tetrahydrofuranyl- dihydrofuranyl-, tetrahydropyranyl-, pyranyl-, thiopyranyl-, aziridinyl-, oxiranyl-, methylenedioxyl-, chromenyl-, isoxazolidinyl-, 1,3-oxazolidin-3-yl- isothiazolidinyl-, 1,3-thiazolidin-3-yl-, 1,2-pyrazolidin-2-yl-, 1,3-pyrazolidin-1-yl-, piperidinyl-, thiomorpholinyl-, 1,2-tetrahydrothiazin-2-yl-, 1,3-tetrahydrothiazin-3-yl-, tetrahydrothiadiazinyl-, morpholinyl-, 1,2-tetrahydrodiazin-2-yl-, 1,3-tetrahydrodiazin-1-yl-, tetrahydroazepinyl-, piperazinyl-, and chromanyl.

9. The compound of claim 1 wherein component W thereof is an optionally substituted histidine residue.

10. A compound selected from the group consisting of:
7-Amino-heptanoic acid {2-(1H-indol-3-yl)-1-[2-(l H-indol-3-yl)-ethylcarbamoyl]-ethyl}-amide;
7-Amino-heptanoic acid [1-[2-(5-fluoro-1H-indol-3-yl)-1-methyl-ethylcarbamoyl]-2-(1H-indol-3-yl)-ethyl]-amide;
7-Amino-heptanoic acid [1-[2-(5-fluoro-1H-indol-3-yl)-ethylcarbamoyl]-2-(1H-indol-3-yl)-ethyl]-amide;
7-Amino-heptanoic acid (2-(1H-indol-3-yl)-1-{2-[2-(4-methoxy-phenyl)-1H-indol-3-yl]-ethylcarbamoyl}-ethyl)-amide;
7-Amino-heptanoic acid [1-(indan-2-ylcarbamoyl)-2-(1H-indol-3-yl)-ethyl]-amide;
7-Amino-heptanoic acid {1-(1H-indol-3-ylmethyl)-2-oxo-2-[4-(2-oxo-2,3-dihydro-benzoimidazol-1-yl)-piperidin-1-yl]-ethyl}-amide;
7-Amino-heptanoic acid [1-[2-(6-fluoro-1H-indol-3-yl)-ethylcarbamoyl]-2-(1H-indol-3-yl)-ethyl]-amide;
7-Amino-heptanoic acid [2-[4-(2-chloro-dibenzo[b,f][1,4]oxazepin-1I -yl)-piperazin-1-yl]-l -(1H-indol-3-ylmethyl)-2-oxo-ethyl]-amide;
6-Amino-hexanoic acid {1-(1H-indol-3-ylmethyl)-2-oxo-2-[4-(2-oxo-2,3-dihydro-benzoimidazol-1-yl)-piperidin-1-yl]-ethyl}-amide;
7-Amino-heptanoic acid (2-(1H-indol-3-yl)-1-{[2-(l H-indol-3-yl)-ethyl]-methyl-carbamoyl}-ethyl)-amide;
7-Amino-heptanoic acid {2-(1H-indol-3-yl)-1-[2-(1H-indol-3-yl)-ethylcarbamoyl]-ethyl}-amide;
7-Amino-heptanoic acid [1-[2-(6-benzyloxy-1H-indol-2-yl)-ethylcarbamoyl]-2-(1H-indol-3-yl)-ethyl]-amide;
6-Amino-hexanoic acid [1-(4-ethyl-benzylcarbamoyl)-2-( 1H-indol-3-yl)-ethyl]-amide;
1-{1-[2-(7-Amino-heptanoyl)-2,3,4, 9-tetrahydro-1H-carbolic-3-carbonyl]-piperidin-4-yl}-1, 3-dihydro-benzoimidazol-2-one;
7-Amino-heptanoic acid (2-(1H-indol-3-yl)-1-{[2-(l H-indol-3-yl)-ethyl]-methyl-carbamoyl}-ethyl)-amide;
7-Amino-heptanoic acid {2-(1H-indol-3-yl)-1-[2-(5-methoxy-1H-indol-3-yl)-ethylcarbamoyl]-ethyl}-amide;
7-Amino-heptanoic acid [1-[4-(biphenyl-4-carbonyl)-piperazine-I -carbonyl]-2-(1H-indol-3-yl)-ethyl]-amide;
7-Amino-heptanoic acid [2-(1H-indol-3-yl)-1-(4-phenyl-butylcarbamoyl)-ethyl]-amide;
7-Amino-heptanoic acid [1-[2-(2-fluoro-phenyl)-ethylcarbamoyl]-2-(1H-indol-3-yl)-ethyl]-amide;
7-Amino-heptanoic acid {1-(1H-indol-3-ylmethyl)-2-oxo-2-[4-(2-oxo-2, 3-dihydro-benzoimidazol-1-yl)-piperid in-1-yl]-ethyl}-amide;
7-Amino-heptanoic acid [2-(1H-indol-3-yl)-1-(1H-indol-5-ylcarbamoyl)-ethyl]-amide;
7-Amino-heptanoic acid [2-(1H-indol-3-yl)-1-(3-phenyl-propylcarbamoyl)-ethyl]-amide;
2-(7-Amino-heptanoylamino)-3-(1H-indol-3-yl)-propionic acid biphenyl-4-ylmethyl ester;
7-Amino-heptanoic acid [2-(1H-indol-3-yl)-1-(2-phenyl-cyclopropylcarbamoyl)-ethyl]-amide;
6-Amino-hexanoic acid [1-(3-ethyl-benzylcarbamoyl)-2-(1H-indol-3-yl)-ethyl]-amide;
7-Amino-heptanoic acid {2-(1H-indol-3-yl)-1-[4-(toluene-4-sulfonyl)-piperazine-1-carbonyl]-ethyl}-amide;
7-Amino-heptanoic acid [1-{4-[4,4-bis-(4-fluoro-phenyl)-butyl]-piperazine-1-carbonyl}-2-(1H-indol-3-yl)-ethyl)-amide;
8-Amino-octanoic acid {1-(1H-indol-3-ylmethyl)-2-oxo-2-[4-(2-oxo-2,3-dihydro-benzoimidazol-1-yl)-piperidin-1-yl]-ethyl}-amide;
6-Amino-hexanoic acid {1-(1H-indol-3-ylmethyl)-2-oxo-2-[4-(2-oxo-2, 3-dihydro-benzoimidazol-1-yl)-piperidin-1-yl]-ethyl}-amide; and
7-Amino-heptanoic acid [2-(1H-indol-3-yl)-1-(2-p-tolyl-ethylcarbamoyl)-ethyl]-amide.

11. A pharmaceutical composition for increasing growth hormone secretion in a mammal, comprising an effective amount of a compound according to claim 1, and a pharmaceutical carrier.

12. A pharmaceutical composition for increasing secretion of gastrin or glucagon in a mammal, comprising an effective amount of a compound according to claim 1, and a pharmaceutical carrier.

13. A pharmaceutical composition for inhibiting the binding of somatostatin to an sst2 receptor, comprising an effective amount of a compound according to claim 1, and a pharmaceutical carrier.

14. A method for increasing growth hormone secretion in a mammal, comprising administering an effective amount of a pharmaceutical composition according to claim 11.

15. A method for increasing secretion of gastrin or glucagon in a mammal, comprising administering an effective amount of a pharmaceutical composition according to claim 12.

16. A method for decreasing somatostatin-induced downregulation of growth hormone secretion in a mammal, comprising administering an effective amount of a pharmaceutical composition according to claim 13.

17. A pharmaceutical composition useful to cause sustained release of growth hormone in a mammal in need thereof, comprising a compound according to claim 1, and a pharmaceutical carrier.

18. A method for facilitating the sustained secretion of growth hormone in a mammal in need thereof, wherein said mammal possesses:
(a) a defect in (1) the expression of the encoding nucleotide sequence for growth hormone, (2) the processing of resultant mRNA, or (3) the translation or intracellular processing and packaging of GH or precursor polypeptide thereof; or
(b) an allele of the growth hormone gene which codes for a growth hormone polypeptide that is insufficiently active;
which comprises administering an effective amount of a pharmaceutical composition according to claim 17.

19. A method for treating a human for one or more symptoms of insufficient growth hormone secretion, wherein said symptom is selected from frailty, hypoglycemia, wrinkled skin, slow skeletal growth, reduced immune function, and reduced organ function, comprising administering an effective amount of a pharmaceutical composition according to claim 11.

20. A method for treating a non-human mammal to enhance the growth and performance thereof, comprising administering an effective amount of a pharmaceutical composition according to claim 11.

21. A pharmaceutical composition according to claim 11 further comprising growth hormone releasing peptide (GHRP) or growth hormone releasing hormone (GHRH).

22. A method for increasing growth hormone secretion in a mammal, comprising administering an effective amount of a pharmaceutical composition according to claim 11, and a further composition comprising growth hormone releasing peptide (GHRP) or growth hormone releasing hormone (GHRH).
